(19) United States
(12) Patent Application Publication
(10) Pub. No.: US 2018/0256730 A1
(43) Pub. Date: Sep. 13, 2018

(54) A POLYMERIC CARRIER FOR DELIVERY OF A PAYLOAD TO A CELL

(71) Applicant: University of Utah Research Foundation, Salt Lake City, UT (US)

(72) Inventors: Kihoon Nam, Salt Lake City, UT (US); Sung Wan Kim, Salt Lake City, UT (US)

(73) Assignee: University of Utah Research Foundation, Salt Lake City, UT (US)

(21) Appl. No.: 15/761,032

(22) PCT Filed: Sep. 16, 2016
(86) PCT No.: PCT/US16/52313

§ 371 Date: Mar. 16, 2018

Related U.S. Application Data
(60) Provisional application No. 62/219,588, filed on Sep. 16, 2015.

(51) Int. Cl. A61K 47/59; A61K 31/7088; A61K 48/00; C08L 79/02

(52) U.S. Cl. CPC A61K 47/59; A61K 31/7088; C08G 73/0253; C08L 79/02; A61K 48/0058

(57) ABSTRACT

A polymeric carrier for delivery of a payload to a cell that can include poly(ethylenimine) and a biodegradable core molecule having disulfide bonds. The carrier can be used to introduce a therapeutic material for gene therapy or other therapeutic purposes.

### PRIORITY DATA

This application claims the benefit of U.S. Provisional Patent Application Ser. No. 62/219,588 filed Sep. 16, 2015, which is incorporated herein by reference.

### BACKGROUND

Gene therapy has the potential to treat many diseases, including cancer, infectious disease, and immune system disorders. However, efficient gene delivery vehicles are a very important factor contributing to the success of gene therapy. Both viral and non-viral delivery systems have been used, but non-viral vectors present a variety of advantages, such as scalability, low immune response, flexible loading capacity, and stability.

Branched poly(ethylenimine) (PEI) 25 kDa is an efficient gene delivery vector with outstanding gene encapsulation efficiency and great endosome escape activity. However, despite these beneficial qualities, it also induces high cytotoxicity. Transfection efficiency and toxicity of PEI are highly dependent upon molecular weight. For example, high molecular weight (HMW) PEI shows high transfection efficiency, but it also induces higher cytotoxicity. In contrast, low molecular weight (LMW) PEI has lower cytotoxicity, but its transfection efficiency is very poor. Therefore, there is a need to develop novel gene delivery vehicles that have high transfection efficiency and low cytotoxicity.

### BRIEF DESCRIPTION OF THE DRAWINGS

FIG. 1 depicts a synthesis scheme and structure of bioreducible poly(ethylenimine) derivatives. i) Cystamine dihydrochloride, ii) methyl acrylate, iii) bPEI 1.8 kDa.

FIGS. 2A-2D depict the ¹H NMR spectrum of example embodiments of a polymer carrier.

FIG. 3 shows the results of an agarose gel electrophoresis retardation assay of PEI derivatives with 0.5 μg pDNA. pDNA only (lane 1), weight ratios of polymer/pDNA=0.25, 5, 1, 2 (lanes 2-5, respectively).

FIGS. 4A-4B depict the zeta potential (A) and size (B) analysis of PEI derivatives/pDNA polyplexes at various weight ratios. PEI 25 kDa (●, [symbol] ), PEI 1.8 kDa (▴, [symbol] ), PEI (-s-s-) 7.6 kDa (▾, [symbol] ), PEI (-s-s-) 16 kDa (▪, [symbol] ), and PEI (-s-s-) 32 kDa (♦, [symbol] ). Results are expressed as mean±standard deviation (n=3).

FIGS. 5A-5F depict the transfection experiment results at various weight ratios in A549 cells (A, B), HeLa cells (C, D), and H9C2 cells (E, F). Results are expressed as mean±standard deviation (n=3).

FIGS. 6A-6C depict the results of a cytotoxicity assay of polyplexes in A549 cells (A), HeLa cells (B), and H9C2 cells (C) by MTT assay. Results are expressed as mean±standard deviation (n=3).

FIG. 7 depicts green fluorescent protein expression levels in A549 cells.

FIGS. 8A-8C depict the results of a cytotoxicity assay in A549 cells (A), HeLa cells (B), and H9C2 cells (C) at various concentrations of PEI derivatives by MTT assay. PEI 25 kDa (●), PEI 1.8 kDa (▴), PEI (-s-s-) 7.6 kDa (▾), PEI (-s-s-) 16 kDa (▪), and PEI (-s-s-) 32 kDa (♦). Results are expressed as mean±standard deviation (n=5).

FIG. 9 depicts the cytotoxicity of rPEI on A549, HT1080, and MCF7 cell viability. Cancer cells were treated with PBS, 25 kDa PEI, 16 kDa rPEI, or 32 kDa rPEI followed by an MTT cell viability assay. Cytotoxicity results were normalized against PBS treated group. The data represent the means+/−SD of triplicate experiments. ***P<0.001 versus 25 kDa PEI.

FIG. 10 illustrates the average size distribution and surface charge of naked Ad, Ad/25 kDa PEI, Ad/16 kDa rPEI, or Ad/32 kDa rPEI. The sizes and charges are the mean+/−SD of five independent experiments. Data describe mean+/−SD. *P<0.05, **P<0.01, ***P<0.001 versus indicated control.

FIG. 11 illustrates the cellular uptake assay of FITC-labeled Ad, Ad-FITC/25 kDa PEI, Ad-FITC/16 kDa rPEI, or Ad-FITC/32 kDa rPEI in A549 and HT1080 cells. The cellular uptake efficiency was measured by FITC intensity. Data describe mean+/−SD. *P<0.05, **P<0.01, ***P<0.001 versus indicated control.

FIGS. 12A-12B depict the transduction efficiency of Ad/GFP, Ad/GFP/25 kDa PEI, Ad/GFP/16 kDa rPEI, or Ad/GFP/32 kDa rPEI in A549, HT1080, and MCF7 cancer cells. FIG. 12A includes representative fluorescence microscopy images of transduced cells. Original magnification: 40×. FIG. 12B illustrates GFP expression levels as quantified by fluorescent analyzer. Data describe mean+/−SD. **P<0.01, ***P<0.001 versus Ad/GFP/25 kDa PEI.

FIG. 13 illustrates the transduction efficiency of Ad/GFP, Ad/GFP/10 kDa PEI, Ad/GFP/25 kDa PEI, Ad/GFP/16 kDa rPEI, or Ad/GFP/32 kDa rPEI in A549 cancer cells. Representative fluorescence microscopy images of transduced cells are shown. Original magnification: 40×. GFP expression level was quantified by fluorescent analyzer. Data describe mean+/−SD ***P<0.001 versus Ad/GFP/25 kDa PEI.

FIG. 14 shows the results of the competition assay of Ad/GFP, Ad/GFP/25 kDa PEI, Ad/GFP/16 kDa rPEI, or Ad/GFP/32 kDa rPEI with CAR specific. A549 cells were pre-incubated with CAR Ab (20, 50 μg/mL), followed by treatment with naked Ad/GFP, Ad/GFP/25 kDa PEI, Ad/GFP/16 kDa rPEI, or Ad/GFP/32 kDa rPEI at 50 MOI. (A) GFP fluorescence microscopy images. GFP expression levels were quantified by fluorescent analyzer. Original magnification: 40×. Data describe mean+/−SD. **P<0.01 versus presence of CAR Ab.

FIGS. 15A-15B show the cancer cell killing effect of naked oAd, oAd/25 kDa PEI, oAd/16 kDa rPEI, or oAd/32 kDa rPEI. (A) Cancer cells were infected with naked oAd, oAd/25 kDa PEI, oAd/16 kDa rPEI, or oAd/32 kDa rPEI at MOI of 5 (A549), 50 (HT1080), and 200 (MCF7), respectively. At 2 days post infection, cell viability was determined by an MTT assay. Data describe mean+/−SD. *P<0.05, **P<0.01, ***P<0.001 versus oAd/25 kDa PEI. (B) The production of infectious Ad viral particles was also measured by limiting dilution assay at 3 days post infection. *P<0.05, **P<0.01 versus oAd/25 kDa PEI.

FIG. 16 depicts measurements of MET or VEGF. Specific knock down of Met or VEGF expression by naked oAd, or oAd/polymers. A549 or HT1080 cells were treated with naked oAd, oAd/25 kDa PEI, oAd/16 kDa rPEI, or oAd/32 kDa rPEI at MOI of 2, 20, respectively. After incubation for three days, each conditioned medium was measured the expression of Met or VEGF by human c-Met or VEGF ELISA assay kit, respectively. Data describe mean+/−SD *P<0.05, **P<0.01, ***P<0.001 versus oAd/25 kDa PEI.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a cell” includes a plurality of such cells.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, a “subject” refers to an animal. In one aspect the animal may be a mammal. In another aspect, the mammal may be a human.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Gene therapy can be used to treat many diseases, such as cancers, infectious diseases, and immune system disorders. Gene therapy incorporates techniques for directly killing diseased cells, producing or inhibiting disease-related proteins, and generally regulating the immune system. One of the most important steps of gene therapy is the efficient delivery of therapeutic genes to target cells of a subject. Both viral and non-viral delivery systems have been used for therapeutic gene delivery. However, non-viral vectors offer several advantages over viral vectors, such as large-scale production, low immune responses, flexible loading capacity of therapeutic genes, and stability of the vector. Hence, the development of efficient and safe non-viral delivery vectors is an important issue in non-viral gene therapy. However, it is still possible to deliver a virus, such as an adenovirus viral vector or other suitable viral vector, to target cells using a non-viral delivery vector or carrier, as will be described in more detail below. And, in some examples, non-viral vectors can also be used to deliver a non-viral payload, as will also be described below. As such the current disclosure provides various methods of treating a disease that includes delivering or administering a therapeutic gene to a target cell of a subject via a non-viral vector or polymeric carrier as described herein.

Typical non-viral vectors include cationic lipids and polymers. Among the cationic polymers, branched poly(ethylenimine) (PEI) 25 kDa can be an effective and inexpensive gene delivery vector. Because of its high amine density, PEI shows outstanding gene encapsulation efficiency and great endosome escape activity. However, it can destabilize the cell membrane and cause cytotoxicity to the cell. Transfection efficiency and toxicity of cationic polymers are highly dependent upon molecular weight and structure. For example, high molecular weight (HMW) PEI shows a high transfection efficiency but, it also induces higher cytotoxicity. Therefore, high toxicity of HMW PEI can limit transfection to in vitro and in vivo conditions. In contrast, low molecular weight (LMW) PEI has lower cytotoxicity, but its transfection efficiency is very poor. Therefore, LMW PEI is not a good candidate as a non-viral gene delivery vector.

Hence, the present inventors have recognized a need for the development of novel gene delivery vectors with both high transfection efficiency and low cytotoxicity. The current technology includes a polymeric carrier for delivery of a payload to a cell. The polymeric carrier can include PEI and a biodegradable core molecule having disulfide bonds. PEI is a synthetic polymer composed of ethylenimine monomers. The chemical structure of PEI is largely divided into linear and branched forms. The linear PEI consists of primary and secondary amines whereas branched PEI contains 3 types of amines with both amine categories of linear PEI as well as tertiary amines. PEI facilitates condensation of nucleic acid such as DNA or siRNA and forms polyplexes at different N/P ratios. Both linear and branched PEI show high transfection efficacy in gene delivery studies. Linear PEI can be more efficient than branched PEI in vivo as less hermetic complexation allows for more efficient dissociation of complexes. Branched PEI shows higher transfection efficacy in vitro due to stronger complexation than linear PEI. Of note, the reducible PEI (rPEI) forms an enhanced complex, which has advantageous attributes of both linear and branched PEI as adenovirus-loaded (or any other suitable viral or non-viral payload) rPEI (Ad/rPEI) can easily dissociate by reducible linkage in vivo while still maintaining stronger condensation properties of branched PEI. This reducible dendritic feature of rPEI makes rPEI a strong candidate for highly efficacious and effective carrier for adenovirus (Ad) (or any other suitable viral or non-viral payload) delivery in vitro and in vivo.

In one aspect, the PEI of the current technology can be branched PEI. In another embodiment, the PEI can have a dendritic feature that allows a complex to form with more profound condensation with lower amount of polymer. This high condensing attribute can enhance adenoviral (or other payload) transduction efficiency as well as reducing adverse effects of the polymer. When bioreducible PEI (i.e. rPEI) gets delivered to the cells, a disulfide bridge of the polymer backbone can easily be cleaved by intracellular glutathione and rapidly be removed from host's or subject's body implying minimized cytotoxicity. Furthermore, dendrimers have other advantages as a non-viral carrier.

Branched PEI can consist of 25, 50 and 25% of primary, secondary, and tertiary amine groups, respectively. Because PEI has many primary amine groups, it can be easily modified to optimize its gene delivery activity and cytotoxicity. As such, there are several approaches that can be used to decrease the toxicity of PEI while maintaining the merits of high transfection efficiency. One way to overcome the cationic polymer cytotoxicity is by combining biodegradable bonds such as ester and disulfide-bonds. However, ester bonds are readily hydrolyzed in an aqueous environment. In contrast, disulfide bonds are not reduced until they are exposed to reducing agents such as β-mercaptoethanol (BME), dithiothritol (DTT), and glutathione (GSH). Therefore, disulfide-linked polymers are more stable than ester-linked polymers in the extracellular environment. In addition, disulfide bonds can be cleaved by glutathione in the intracellular cytoplasm.

Therefore, to overcome the limitations of current PEI payload delivery systems, a bioreducible PEI (PEI (-s-s-) or rPEI) can be used to impart biodegradable characteristics while maintaining amine density. Therefore, the current technology can include a biodegradable core molecule having disulfide bonds. In one aspect, the biodegradable core molecule can have biodegradable bonds that are exclusively disulfide bonds. In another aspect, greater than 50% of the biodegradable bonds in the biodegradable core molecule are disulfide bonds. In another aspect, greater than 75% of the biodegradable bonds in the biodegradable core molecule are disulfide bonds. In another aspect, greater than 90% of the biodegradable bonds in the biodegradable core molecule are disulfide bonds. In some examples, the biodegradable core molecule can include from 1 to 13 disulfide bonds.

The biodegradable core molecule can also include a number of other chemical bonds or functional groups, such as amide groups, tertiary amines, ester groups, and/or other suitable bonds or functional groups. In one specific example, the biodegradable core molecule can include from 4 to 28 amide bonds. In yet another specific example, the biodegradable core molecule can include from 2 to 14 tertiary amines. In one aspect, the biodegradable core molecule can be a dendrimer. In some examples, the dendrimer can have from 4 to 16 termini. In some examples, the dendrimer can have 8 termini. In some further examples, each of the termini can be an amino group. One or more PEI polymers can attach to the biodegradable core molecule at the termini. In some examples, the dendrimer can have a molecular weight of from about 400 daltons (Da) to about 3500 Da.

Further, the bioreducible core molecule allows for increased molecular weight while reducing the cytotoxicity of the copolymer. To achieve such a bioreducible PEI (rPEI), LMW PEI can be connected to a biodegradable core molecule containing disulfide bonds. The LMW PEI can have any suitable molecular weight. In one aspect, the PEI can have a molecular weight of between 1.0 and 3.0 kDa. In another aspect, the PEI can have a molecular weight of 1.6 to 2.0 kDa. More specifically, bioreducible cationic copolymers can include a PEI of about 1.8 kDa and a dendritic core molecule containing disulfide bonds. Such a bioreducible poly(ethylenimine) (rPEI), derived from low molecular weight PEI (about 1.8 kDa), can be more efficient for gene delivery than current PEI gene delivery systems.

Generally, the molecular weight and charge density of polymeric carrier affects the cytotoxicity and gene transfer activity. For example, enhanced transfection efficiency can result from an increased molecular weight and decreased cytotoxicity can result from the degradation of the core molecule in the intracellular cytoplasm. The current technology increases the molecular weight of the PEI by connecting it with a disulfide-containing core molecule that allows for biodegradation. The polymeric carrier can have any suitable molecular weight. In one aspect, the polymeric carrier can have a molecular weight of between 7.4 and 7.8 kDa. In one aspect, the polymeric carrier can have a molecular weight of between 15.5 and 16.5 kDa. In one aspect, the polymeric carrier can have a molecular weight of between 31.5 and 32.5 kDa. In one aspect, the polymeric carrier can have a molecular weight of between 7.4 and 32.5 kDa. In one aspect, the polymeric carrier can have a molecular weight of about 7.6 kDa. In one aspect, the polymeric carrier can have a molecular weight of about 16 kDa. In one aspect, the polymeric carrier can have a molecular weight of about 32 kDa. Further, in some examples, the polymeric carrier can have a polydispersity of from about 1.20 to about 1.26.

Such polymeric carriers can have higher transfection efficiency, as compared to non-degradable PEI 25 kDa, 1.8 kDa, Lipofectamine®, and FuGENE® 6. In one aspect, the polymeric carrier can have a transfection efficiency of greater than 60%. In one aspect, the polymeric carrier can have a transfection efficiency of greater than 80%. Moreover, the rPEI derivatives can have relatively lower cytotoxicity than Lipofectamine® 2000 and PEI 25 kDa in various cell types, due to the degradability of the polymers. In addition, these polymers can have great plasmid condensing ability. In one embodiment, the polymeric carrier can be multi degradable bioreducible core-crosslinked polyethyleneimine (rPEI) (i.e. reducible PEI) polymer. In some embodiments, the rPEI can have low immunogenicity and toxicity while having higher transduction efficacy and stability.

As previously discussed, the polymeric carrier can include a payload. Any suitable payload can be used. For example, the payload can include at least one of DNA, RNA, a protein, a virus, and a therapeutic agent. In another aspect the payload can be a member selected from the group consisting of DNA, RNA, a protein, a virus, and a therapeutic agent. In one aspect, the payload can be used to replace a mutated gene. In another aspect, the payload can be used to inactivate or inhibit an improperly functioning gene. In another aspect, the payload can be used to introduce a new gene into a cell. In a further aspect, the payload can be a vector, such as an adenoviral vector.

The size of the payload can be very important in maintaining low cytotoxicity. For example, if the payload or the ratio of payload to polymer carrier becomes too large, it can increase the cytotoxicity of the polymeric carrier. Further it can adversely affect the transfection efficiency. The payload can have any suitable size or molecular weight to maintain good transfection efficiency and low cytotoxicity. In one aspect, the payload can have a molecular weight of up to 100 kDa. In one aspect, the payload can have a molecular weight of up to 50 kDa. In one aspect, the payload can have a molecular weight of up to 20 kDa. In one aspect, the payload can have a molecular weight of up to 10 kDa. In one aspect, the weight ratio of payload to polymeric carrier can be from about 1:1 to about 1:10. In another aspect, the weight ratio of payload to polymeric carrier can be from about 1:1 to about 1:8. In another aspect, the weight ratio of payload to polymeric carrier can be from about 1:1 to about 1:4. In another aspect, the weight ratio of payload to polymeric carrier can be from about 1:1 to about 1:2.

Despite the disadvantages of using polymeric carriers or polymeric vectors alone and the disadvantages of using typical viral vectors alone, the present inventors have discovered that in one embodiment, a hybrid vector (i.e. polymer vector and viral vector combined) can pose a number of specific advantages, including improved payload delivery and efficacy. In this regard, typical cationic polymers including poly(ethyleneimine) (PEI), poly-L-lysine (PLL), and liposomes/lipids can be complexed with Ad, or other suitable viral vectors, resulting in enhanced transduction by charge mediated internalization into host cells which consequently overcomes coxsackievirus and adenovirus receptor (CAR) dependency of adenoviral vectors.

One particular application of polymer-payload complexes (including polymer-viral vector complexes) of the present invention is in the treatment of cancer. rPEI's can be synthesized at different molecular weights and complexed with adenovirus (Ad) or other vectors at varying molar ratios to optimize delivery of Ad/polymer complex.

In some embodiments, Ad/rPEIs can provide significantly enhanced transduction efficiency compared to either naked Ad or Ad/25 kDa PEI in both coxsackievirus and adenovirus receptor (CAR) positive and negative cancer cells. In one embodiment, cellular uptake of relatively small size of Ad/16 kDa rPEIs (below 200 nm) can be more critical to a complex's internalization than its surface charge. Cancer cell killing effect and viral production can be significantly increased when oncolytic Ad (RdB/shMet, or oAd) is complexed with 16 kDa rPEI in comparison to naked oAd, oAd/25 kDa PEI, or oAd/32 kDa rPEI treated cells. This increased anticancer cytotoxicity is more readily apparent in CAR negative MCF7 cells and can be used to treat a broad range of cancer cells. Furthermore, A549 and HT1080 cancer cells treated with oAd/16 kDa rPEI can have significantly decreased Met and VEGF expression compared to either naked oAd or oAd/25 kDa PEI. Overall, shMet expressing oncolytic Ad complexed with multi degradable bioreducible core-crosslinked PEI can be used as efficient and safe cancer gene therapy.

Further, a novel multi-biodegradable and bioreducible core-crosslinked PEI with high condensation capacity and surface charge can be designed and synthesized that results in highly efficacious Ad delivery to cancer cells. This polymer can have high gene transduction ability with low cytotoxicity due to utilization of low molecular weight 1.8 kDa PEI and reductive function from disulfide linkage in the polymer backbone. Surface charge of Ad/rPEIs can be greater than or equal to 20 mV, which is similar than the surface charge of highly cationic 25 kDa PEI, while having significantly smaller size than 25 kDa PEI, which can have significant contribution toward higher cellular uptake by Ad complexed with rPEIs. Importance of size over charge is further supported by Ad/16 kDa rPEI showing higher therapeutic efficacy than Ad/32 kDa rPEI as Ad/32 kDa rPEI can have more cationic surface charge but still be less effective than Ad/16 kDa rPEI due to approximately 100 nm higher diameter. The rPEI can be complexed with Ad at higher condensation capacity, which can significantly reduce polymer dose in clinical application. The Ad/rPEI complex can exhibit increased transduction efficiency and can be internalized by CAR-independent pathway with minimal toxicity. Ad complexed with rPEI can result in significantly augmented cancer cell killing effect and therapeutic gene expression by oncolytic Ad than naked Ad or Ad/25 kDa PEI. Hence, the multi-degradable bioreducible PEI coated oncolytic Ad of the current technology can be used as a therapeutic agent in the treatment of malignant cancers.

### EXAMPLES

The materials used in the following examples can be obtained from any suitable source. As non-limiting examples, branched polyethylenimine (bPEI 1.8 kDa, Mw 1,800 Da) can be purchased from Polysciences (Warrington, Pa.). Branched polyethylenimine (bPEI 25 kDa, Mw 25,000 Da), methanol (MeOH), cystamine dihydrochloride, triethylamine (TEA), methyl acrylate (MA), magnesium sulfate, diethyl ether, and thiazolyl blue tetrazolium bromide (MTT) can be purchased from Sigma-Aldrich (St. Louis, Mo.). A BCA protein assay kit can be purchased from Pierce (Rockford, Ill.). The Luciferase assay system, reporter lysis buffer, and FuGENE® 6 can be purchased from Promega (Madison, Wis.). Dulbecco's modified Eagle's medium (DMEM), Opti-MEM®, Dulbecco's phosphate buffered saline (DPBS), TrypLE™ Express, SYBR safe DNA gel stain, and Lipofectamine® 2000 can be purchased from Invitrogen (Carlsbad, Calif.). Fetal bovine serum (FBS) can be purchased from Seradigm (Radnor, Pa.). Dialysis membranes can be purchased from Spectrum Laboratories (Rancho Dominguez, Calif.).

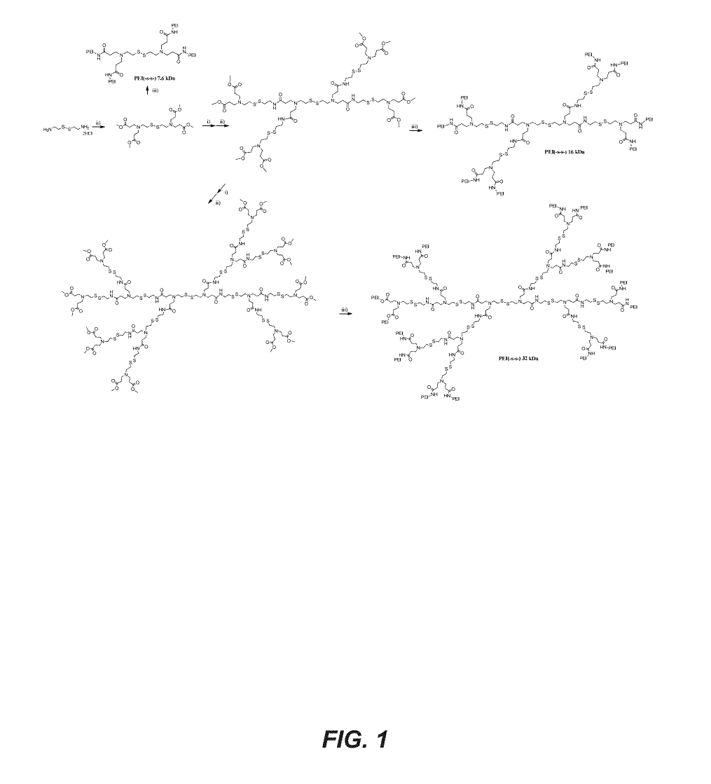

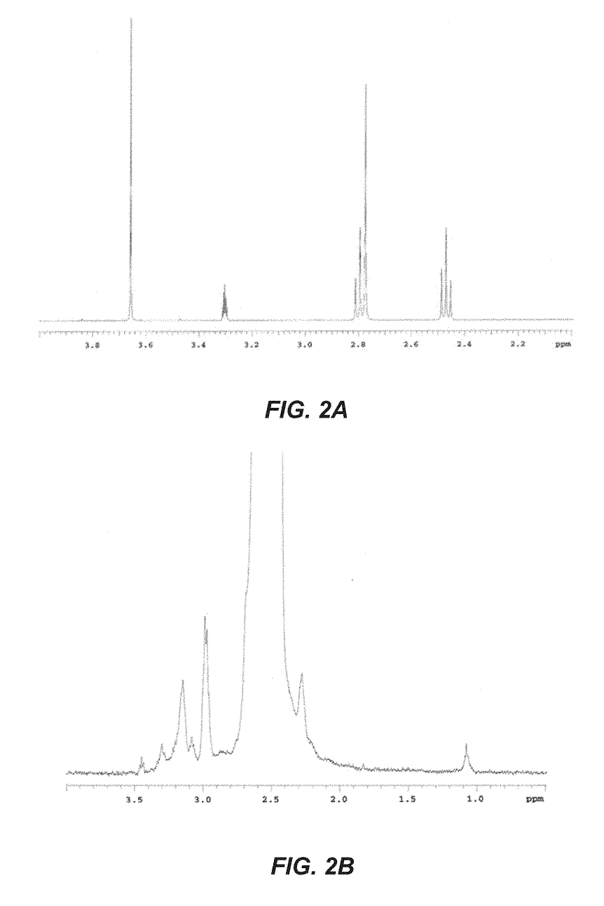

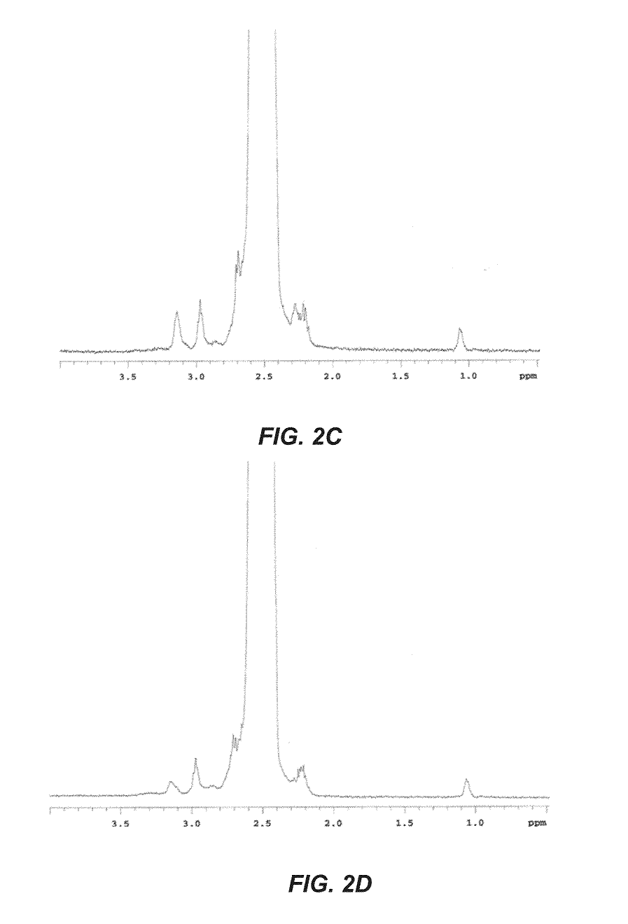

FIG. 1 shows the molecular structure of the bioreducible poly(ethylenimine) derivatives described in each of Examples 1-3. Michael addition-amidation reactions were performed repeatedly for the synthesis of core molecules containing disulfide bonds. After synthesizing the dendritic core molecule, bPEI 1.8 kDa was conjugated with core molecule. Each reaction step was monitored by Thin-Layer Chromatography (TLC) and ¹H NMR. For ¹H NMR analysis, the samples were dissolved either in MeOD or D2O. Because of the unique ¹H NMR peak for methoxy group of methyl acrylate (δ 3.66), reactions were easy to identify. The ¹H NMR results of higher molecular weight polymers showed similar patterns with a 7.6 kDa polymer. The ¹H NMR spectrum of the each polymer is illustrated in FIGS. 2A-2D.

Cystamine dihydrochloride (400 MHz, D₂O)
δ 3.05 (—SCH₂CH₂NH₃⁺), δ 3.42 (—SCH₂CH₂NH₃⁺)
1^{st} core molecule (400 MHz, MeOD)
δ 2.48 (—NCH₂CH₂CO—), δ 2.80 (—SCH₂CH₂N—, —NCH₂CH₂CO—), δ 3.66 (—O—CH₃)
Bioreducible PEI (PEI (-s-s-) or rPEI) 7.6 kDa (400 MHz, D₂O)
δ 2.28 (—NCH₂CH₂CO—; core), δ 2.45˜2.75 (—NCH₂CH₂N—; PEI), δ 2.98˜3.38 (—SCH₂CH₂N—, —NCH₂CH₂CO—; core)

The molecular weight was calculated from ¹H NMR spectra and gel permeation chromatography (GPC) data. Based on the results, one bPEI 1.8 kDa had been conjugated to each of the primary amine branches of the dendritic core molecule. The molecular weight of the bioreducible poly(ethylenimine) derivatives was estimated to be 7.6 kDa (P.D.=1.20), 16 kDa (P.D.=1.26), and 32 kDa (P.D.=1.24), respectively. P.D. values of step polymerization reactions were typically around 2.0. However, P.D. values of PEI (-s-s-) derivatives were about 1.26. This represents that the PEI (-s-s-) derivatives have high reproducibility.

### Example 1—Synthesis of PEI (-s-s-) 7.6 kDa

Methyl acrylate (0.213 mol) was mixed with 10 mL of MeOH. Cystamine dihydrochloride (8.881 mmol) and TEA (0.018 mol) were dissolved in 20 mL of MeOH and cystamine solution was added dropwise to methyl acrylate solution over 30 min. After two days of reaction at room temperature under a nitrogen atmosphere, MeOH and TEA were removed by evaporation by heating to 40° C. and stirring for 30 minutes. A viscous light yellow liquid interspersed with white precipitate was dissolved in diethyl ether and extracted with water. Diethyl ether layers were dried over magnesium sulfate, filtered, and evaporated (yield: 82.97%, theoretical mass: 496.6 Da) to obtain a 1^{st} core molecule.

bPEI 1.8 kDa (8.055 mmol) was mixed with 50 mL of MeOH. The 1^{St} core molecule (0.201 mmol) was dissolved in 20 mL of MeOH and the 1^{st} core molecule solution was added to the bPEI solution dropwise over 30 min. After three days of reaction at room temperature under a nitrogen atmosphere, MeOH was removed by evaporation. A viscous liquid was dissolved in water and dialyzed (MWCO=1000 Da for 2 days, and MWCO=3500 Da for 1 day). Then, the PEI (-s-s-) 7.6 kDa was filtered, and lyophilized (yield: 68.40%).

### Example 2—Synthesis of PEI (-s-s-) 16 kDa

Cystamine dihydrochloride (0.040 mol) and TEA (0.081 mol) were dissolved in 100 mL of MeOH. The 1^{st} core molecule, described in Example 1, (1.007 mmol) was dissolved in 50 mL of MeOH and the 1^{st} core molecule solution was added to cystamine solution dropwise over 30 min. After four days of reaction at 4° C. under a nitrogen atmosphere, MeOH and TEA were removed by evaporation. The mixture was dissolved in water and dialyzed (MWCO=500 Da for 4 days). Then, the sample was lyophilized (yield: 50.72%, theoretical mass: 977.6 Da).

Methyl acrylate (0.068 mol) was mixed with 12 mL of MeOH. The lyophilized sample (0.853 mmol) and TEA (6.824 mmol) were dissolved in 8 mL of MeOH and sample solution was added dropwise over 30 min. After two days of reaction at room temperature under a nitrogen atmosphere, MeOH and TEA were removed by evaporation by heating to 40° C. and stirring for 30 minutes. A 2^{nd} core molecule was extracted with diethyl ether and evaporated (yield: 74.66%, theoretical mass: 1666.3 Da)

bPEI 1.8 kDa (9.602 mmol) was mixed with 50 mL of MeOH. The 2^{nd} core molecule (0.120 mmol) was dissolved in 20 mL of MeOH and the 2^{nd} core molecule solution was added to bPEI solution dropwise over 30 min. After five days of reaction at room temperature under a nitrogen atmosphere, MeOH was removed by evaporation. A product was dissolved in water and dialyzed (MWCO=3500 Da for 3 days and MWCO=10,000 Da for 1 day). Then, the PEI (-s-s-) 16 kDa was filtered and lyophilized (yield: 58.74%).

### Example 3—Synthesis of PEI (-s-s-) 32 kDa

Cystamine dihydrochloride (0.048 mol) and TEA (0.096 mol) were dissolved in 100 mL of MeOH. The 2^{nd} core molecule, described in Example 2, (0.600 mmol) was dissolved in 50 mL of MeOH and the 2^{nd} core molecule solution was added to cystamine solution dropwise over 30 min. After four days of reaction at 4° C. under a nitrogen atmosphere, MeOH and TEA were removed by evaporation. The mixture was dissolved in water and dialyzed (MWCO=500 Da for 3 days and MWCO=1000 Da for 3 hours). Then, the sample was lyophilized (yield: 47.55%, theoretical mass: 2628.2 Da).

Methyl acrylate (0.053 mol) was mixed with 12 mL of MeOH. The lyophilized sample (0.329 mmol) and TEA (5.264 mmol) were dissolved in 8 mL of MeOH and the sample solution was added dropwise over 30 min. After three days of reaction at room temperature under a nitrogen atmosphere, MeOH and TEA were removed by evaporation by heating to 40° C. and stirring for 30 minutes. A 3^{rd} core molecule was extracted with diethyl ether and evaporated (yield: 72.45%, theoretical mass: 4005.7 Da)

bPEI 1.8 kDa (7.987 mmol) was mixed with 50 mL of MeOH. The 3^{rd} core molecule (0.050 mmol) was dissolved in 20 mL of MeOH and the 3^{rd} core molecule solution was added to bPEI solution dropwise over 30 min. After five days reaction at room temperature under a nitrogen atmosphere, MeOH was removed by evaporation. A product was dissolved in water and dialyzed (MWCO=3500 Da for 3 days and MWCO=10,000 Da for 1 day). Then, the product was filtered and lyophilized (yield: 52.69%).

### Example 4—Determining Polyplex Formation

The formation of nano-sized polyplexes with a positive charge is an important factor in polymeric gene delivery. To confirm the polyplex formation, polyplexes were prepared at various weight ratios ranging from 0.25 to 2. Non-degradable branched PEI 25 kDa and 1.8 kDa were used as controls.

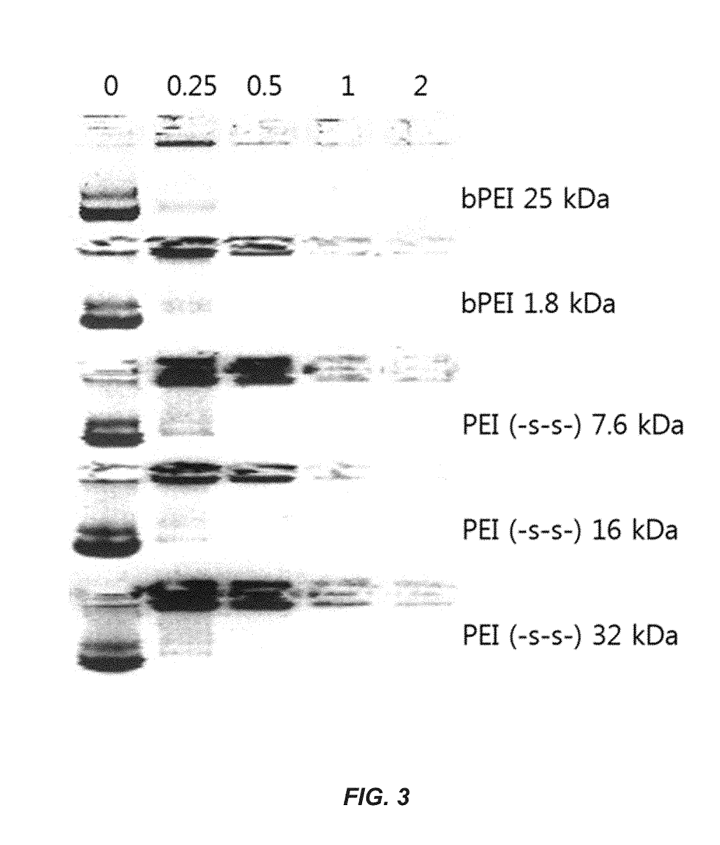

First, the self-assembly of the synthesized polymers with pDNA was investigated by gel retardation assay at 37° C. 0.5 μg of plasmid DNA (pDNA, gWiz-Luc) was mixed with PEI derivatives in 10 μL of HEPES buffered saline (10 mM HEPES, 1 mM NaCl, pH 7.4) at various weight ratios, and incubated 30 min. After 30 min of incubation at room temperature, 2 μL of loading dye was added to each sample and then the samples were analyzed by electrophoresis on a 0.7% agarose gel plate containing SYBR safe gel staining solution. As shown in FIG. 3, all synthesized polymers showed a complete retardation of pDNA at a weight ratio of 0.5.

Next, the particle size and the surface charge of polyplexes were measured to determine the condensing ability of the synthesized polymers. The average size and zeta-potential of each sample of bioreducible PEI derivatives with 4 μg pDNA (gWiz-Luc) at 25° C. were examined using the Nano ZS (Malvern Instruments Ltd., Worcestershire, UK) with a He—Ne laser (633 nm). 100 μL of polyplex solutions were prepared in HEPES buffered saline (10 mM HEPES, 1 mM NaCl, pH 7.4) at various weight ratios ranging from 0.25 to 2, respectively. After 30 min of incubation, sample solutions were diluted to a final volume of 600 μL before the measurement.

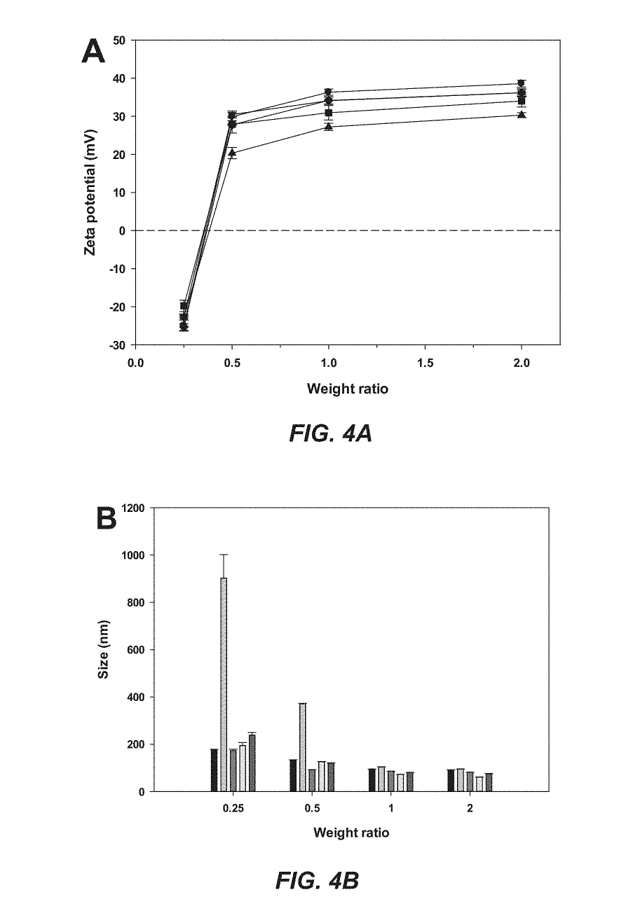

The surface charge increased with increasing weight ratios for all polymers (FIG. 4A). Surface charge of the PEI (-s-s-) 7.6 kDa polyplex (36.20±0.93 mV), the PEI (-s-s-) 16 kDa polyplex (34.03±1.56 mV), and the PEI (-s-s-) 32 kDa polyplex (36.17±1.06 mV) were stronger than the bPEI 1.8 kDa (30.32±0.66 mV) and weaker than the bPEI 25 kDa (38.57±0.90 mV) at weight ratio of 2. In addition, the particle size decreased with increasing weight ratios (FIG. 4B). The particle sizes of PEI (-s-s-) polyplexes (7.6 kDa: 80.95±0.30 nm, 16 kDa: 61.04±0.14 nm, and 32 kDa: 74.35±1.99 nm) were smaller than bPEI 25 kDa (90.82±1.30 nm) and 1.8 kDa (94.22±0.86 nm). These results represent that the condensing ability of PEI (-s-s-) was better than non-degradable branched bPEI 25 kDa and 1.8 kDa.

### Example 5—Transfection Efficiency and Cytotoxicity of Polyplexes

Cell Culture.

Human lung adenocarcinoma epithelial cell line (A549), human cervical cancer cell line (HeLa), and rat cardiomyoblast cell line (H9C2) were grown in DMEM containing 10% FBS. The cells were maintained on plastic tissue culture dishes at 37° C. in a humidified atmosphere containing 5% CO2.

Luciferase Expression and Cytotoxicity.

3.0×10⁴ cells were seeded in 24-well plates in 500 μL of medium (DMEM) containing 10% FBS for a day before transfection. 0.5 μg of pDNA (gWiz-Luc) was complexed with PEI (-s-s-) derivatives in 50 μL of serum free medium (Opti-MEM®) and incubated for 30 min at room temperature. Lipofectamine® 2000 and FuGENE® 6 were used as controls (incubation time: 15 min). The medium was replaced by 450 μL of serum free medium (DMEM) and the polyplexes were added. Following four hours treatment of polyplexes, the medium was replaced by 500 μL of fresh medium (DMEM) containing 10% FBS, and plates were incubated for 2 days at 37° C.

For the luciferase assay, the cells were washed with DPBS and lysed with 200 μL of reporter lysis buffer for 30 min at room temperature. The luciferase activity of 20 μL cell lysate was evaluated by using Tecan Infinite M200 Pro spectrophotometer (Tecan Group Ltd., Männedorf, Switzerland) and the total protein concentration was measured by using a Micro BCA assay reagent kit (Pierce, Rockford, Ill.).

For cytotoxicity assay of polyplexes, 20 μL of filtered MTT solution (2 mg/mL in DPBS) was added to each well and incubated further for 2 h. After incubation, the medium was removed from the well and 500 μL DMSO was added to dissolve insoluble formazan crystals. The absorbance was measured at 565 nm using a Tecan Infinite M200 Pro spectrophotometer and the cell viability was calculated as a percentage relative to untreated control cells. All experiments were performed in triplicate.

GFP Expression.

A549 (1.0×10⁵ cells/well) cells were seeded in 12-well plates with 1 mL of medium (DMEM) containing 10% FBS for a day before transfection. 1.0 μg of pDNA (gWiz-GFP) was complexed with PEI (-s-s-) derivatives in 100 μL of serum free medium (Opti-MEM®), and incubated for 30 min at room temperature. Lipofectamine® 2000 and FuGENE® 6 were used as controls (incubation time: 15 min). The medium was replaced by 900 μL of serum free medium (DMEM), and polyplex was added. After four hours of incubation, the cells were washed with DPBS and the medium was replaced by 1 mL of fresh medium (DMEM) containing 10% FBS, and then incubated further for 2 days at 37° C. The GFP expression was evaluated by using an EVOS microscope (AMG, Bothell, Wash.).

Results.

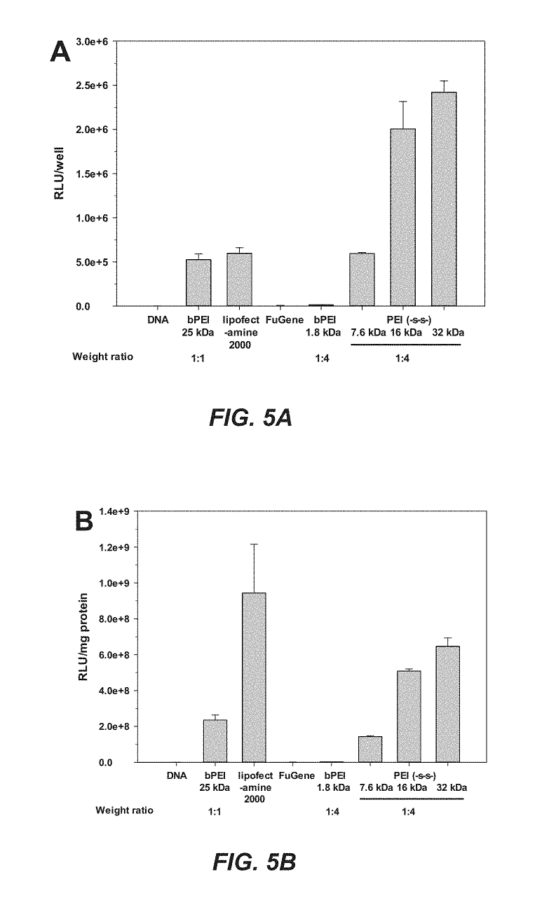

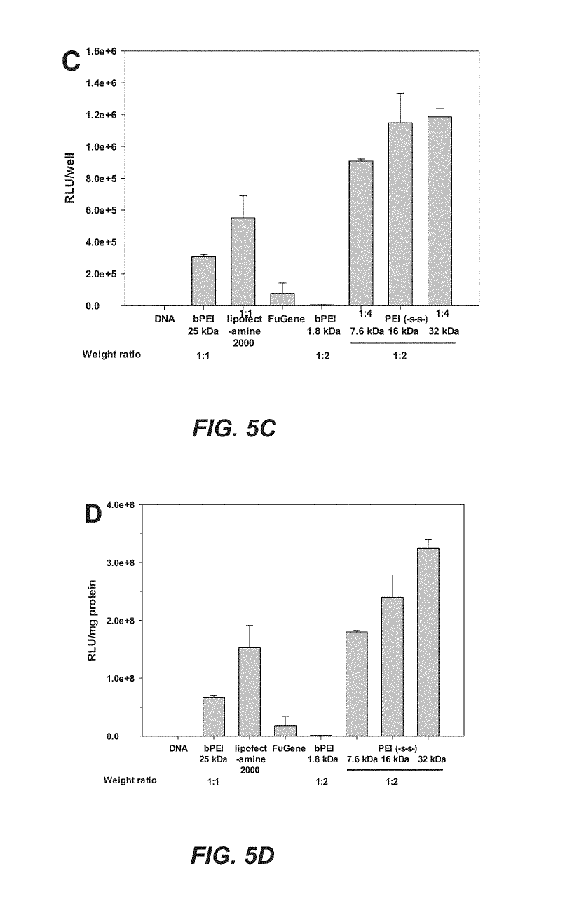

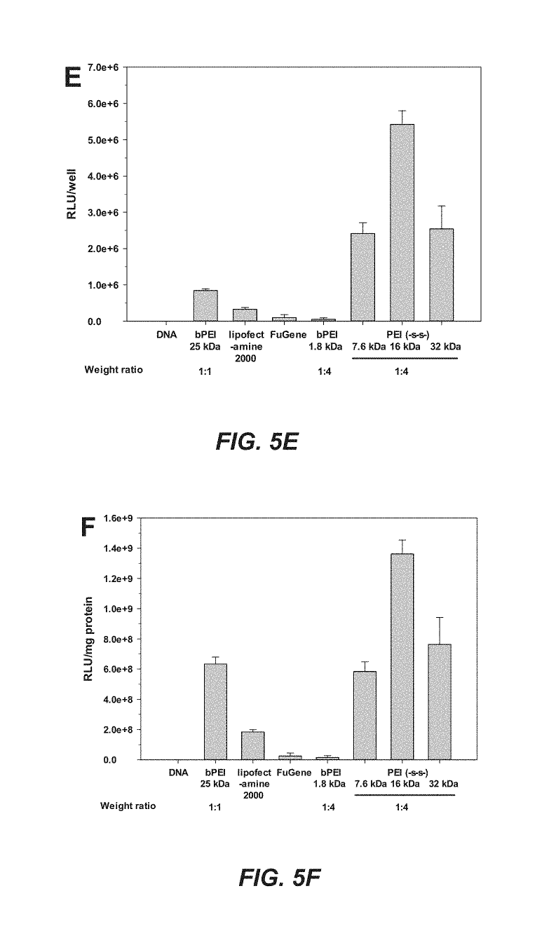

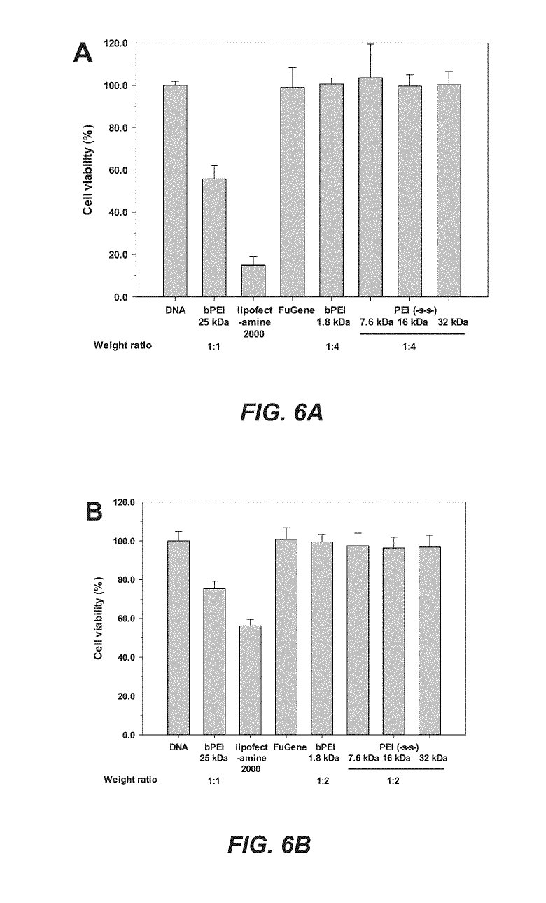

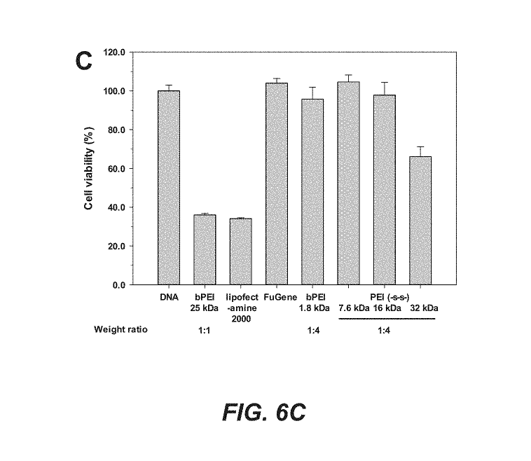

The transfection efficiency of PEI (-s-s-) derivatives was evaluated in the A549, HeLa, and H9C2 cells using luciferase (gWiz-Luc) and GFP (gWiz-GFP) genes. bPEI 25 kDa (1 μg per 1 μg pDNA), bPEI 1.8 kDa (2 or 4 μg per 1 μg pDNA), Lipofectamine® 2000 (2 μL per 1 μg pDNA), and FuGENE® 6 (1.5 μL per 1 μg pDNA) were used as controls. In order to find the optimal gene transfer condition, the transfection experiments were performed at various weight ratios (data not shown). Whereas bPEI 1.8 kDa did not show the transfection efficiency, PEI (-s-s-) derivatives showed higher transfection efficiency at weight ratio of 4 for A549 and H9C2 cell line, and 2 for HeLa cell line. Due to the cytotoxicity of polyplexes (FIGS. 6A-6C), the total amount of cellular protein from Lipofectamine® 2000 and PEI 25 kDa treated groups were much lower than other polymer treated groups. Therefore, Lipofectamine® 2000 showed the highest transfection efficiency in A549 cell line (FIG. 5B). In order to prevent misinterpretation of results, the transfection efficiency was reported both RLU/well (5A, 5C, 5E) and RLU/mg protein (5B, 5D, 5F). In the A549 cell line, PEI (-s-s-) 16 kDa and 32 kDa showed similar transfection efficiency at weight of 4 (FIG. 5A). The order of transfection efficiency was PEI (-s-s-) 32 kDa≥PEI (-s-s-) 16 kDa» Lipofectamine® 2000≈PEI (-s-s-) 7.6 kDa≈bPEI 25 kDa>FuGENE® 6≈bPEI 1.8 kDa (RLU/well). The transfection efficiency of PEI (-s-s-) polyplexes followed a similar order in the HeLa cell line (FIG. 5C, 5D). However, the transfection efficiency of PEI (-s-s-) 32 kDa was lower than PEI (-s-s-) 16 kDa in the H9C2 cell line (FIG. 5E, 5F) due to their cytotoxicity (FIG. 6C). As a result, PEI (-s-s-) derivatives exhibited an improved transfection efficiency in all three cell lines. Especially, PEI (-s-s-) 16 kDa gave 3.6 times higher gene expression than bPEI 25 kDa in HeLa cell and 7.4 times higher than Lipofectamine® 2000 in H9C2 cell.

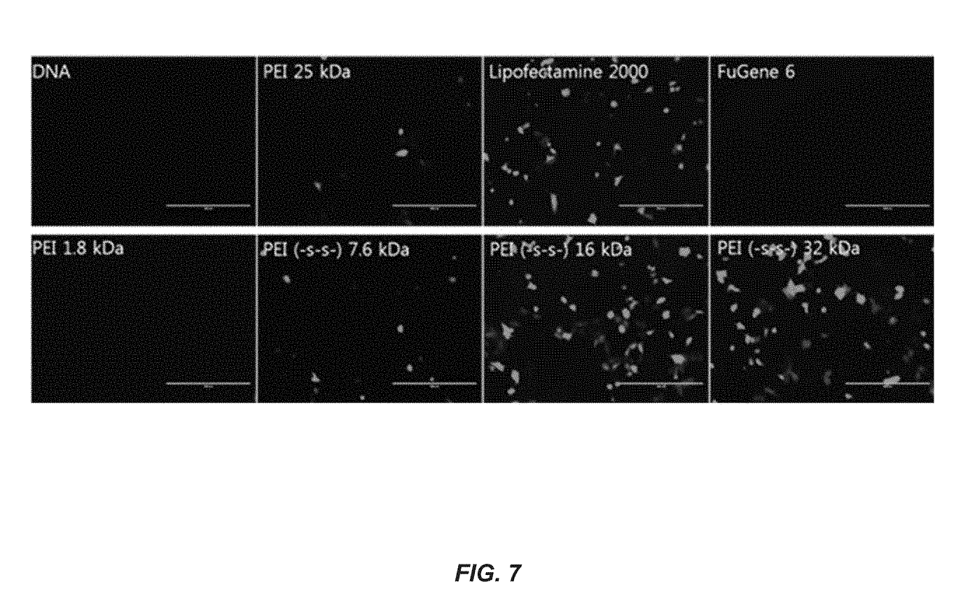

Transfection results with GFP gene showed similar results in A549 cells (FIG. 7). PEI (-s-s-) 16 kDa and 32 kDa showed higher transfection efficiency than other polymers at a weight ratio of 4. The same result was observed in other cells (data not shown).

The cytotoxicity of polyplexes is a very important factor in polymeric gene delivery. The cytotoxicity of polyplexes are highly correlated with their surface charge and molecular weight. Generally, polyplex toxicity increases with increasing surface charge and molecular weight. However, PEI (-s-s-) derivatives exhibited a relatively low cytotoxicity due to their biodegradability (FIGS. 6A-6C).

### Example 6—Cytotoxicity of Polymers

Cells were seeded at 5000 cells/well in 96-well plates in 90 μL of DMEM containing 10% FBS and incubated at 37° C. for one day. Then 10 μL of polymer solution at various concentrations was added and cells were incubated for 2 days before assay.

To measure the cytotoxicity of PEI (-s-s-), MTT assays were performed. 10 μL of filtered MTT solution (2 mg/mL in DPBS) was added to each well and incubated further for 2 h. After incubation, the medium was removed from the well and 100 μL DMSO was added to dissolve insoluble formazan crystals. The absorbance was measured at 565 nm using a Tecan Infinite M200 Pro spectrophotometer and the cell viability was calculated as a percentage relative to untreated control cells. All experiments were performed in quintuplicate.

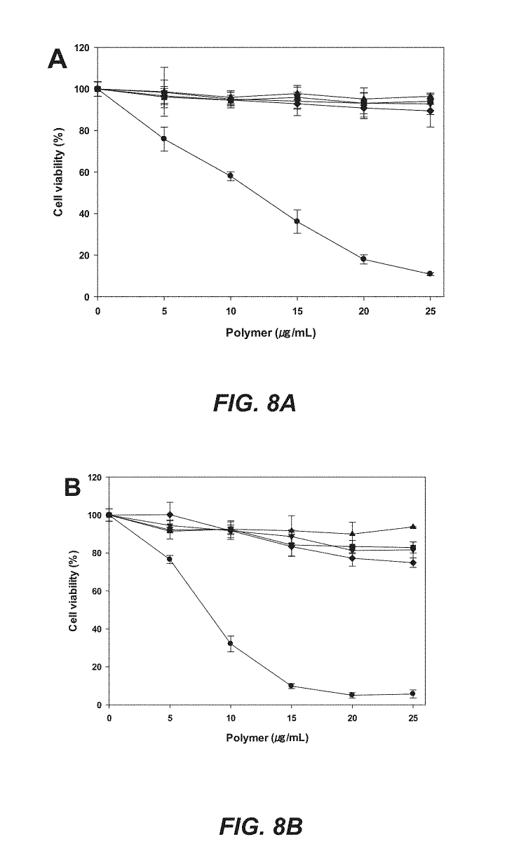

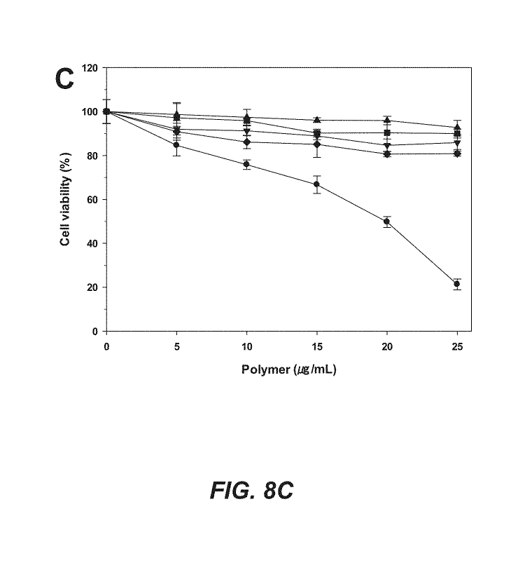

The cytotoxicity of polymers was evaluated in the A549, HeLa, and H9C2 cells by MTT assay (FIGS. 8A-8C). bPEI 25 kDa and 1.8 kDa were used as controls. Normally, cell viability tends to decrease with increasing molecular weight. Therefore, PEI (-s-s-) derivatives showed slightly increased cytotoxicity compared to PEI 1.8 kDa. The order of cell viability was bPEI 1.8 kDa>PEI (-s-s-) 7.6 kDa>PEI (-s-s-) 16 kDa>PEI (-s-s-) 32 kDa» bPEI 25 kDa in all three cells. However, PEI (-s-s-) derivatives were almost non-toxic, while the relative cell viability with bPEI 25 kDa was below 20%. Moreover, despite the higher molecular weight, PEI (-s-s-) 32 kDa showed lower cytotoxicity than bPEI 25 kDa due to its biodegradability. From the results, we observed that bPEI 25 kDa was highly toxic and PEI (-s-s-) derivatives were much less toxic in all three cell lines.

### Example 7—Evaluation of Polymers for Cancer Gene Therapy

7.1 Cell Lines and Adenoviruses

The human cancer (A549 (lung carcinoma), HT1080 (fibrosarcoma), MCF7 (breast adenocarcinoma), and human embryonic kidney (HEK) 293 cells were purchased from the American Type Culture Collection (ATCC, Manassas, Va.) and maintained in Dulbecco's modified Eagle's Media (DMEM; Gibco-BRL, Grand Island, N.Y.) containing 10% fetal bovine serum (FBS; Gibco-BRL) in 37° C. incubator with 5% CO2. The Ad/GFP virus is an E1 region-deleted, replicating-incompetent Ad expressing green fluorescent protein (GFP) under the control of the cytomegalovirus promoter (Ad/GFP). RdB/shMet (oAd) is a shMet expressing oncolytic Ad that replicates under the control of E1A, and E1B double mutated cancer-specific promoter. Ads were propagated in HEK 293 cells and purified by CsCl gradient ultra-centrifugation. The number of viral particles (VP) was calculated from the optical density measurement at 260 nm, which 1 absorbency unit is equivalent to 1.1×10¹² viral particles per mL.

7.2 Preparation of PEI(s-s)

### 1st Cystamine Core

1 g of cystamine dihydrochloride (4.44 mmol) was dissolved in 10 mL of MeOH, and 1.24 mL of TEA (0.009 mol) was added into the cystamine solution. The unsolved cystamine was removed by filtration. 9.5 mL of methyl acrylate (0.107 mol) was mixed with 10 mL of MeOH and then cystamine solution was slowly added to the methyl acrylate solution over 30 min. The solution was reacted for 2 days at room temperature under a nitrogen atmosphere. After 2 days, to remove the MeOH and TEA, the solution was evaporated. The white precipitate with the interspersed viscous light yellow liquid was dissolved in diethyl ether and extracted with water. After extraction, the 1st cystamine core in diethyl ether was obtained by evaporation.

### 2nd Cystamine Core

10.9 g of cystamine dihydrochloride (48.33 mmol) and 13.5 mL of TEA (0.097 mol) were dissolved in 100 mL of MeOH. 600 mg of 1st cystamine core (1.007 mmol) was dissolved in 5 mL and then slowly dropped to cystamine solution over 30 min. The solution was reacted for 4 days at 4° C. under a nitrogen atmosphere. After reaction, the mixture was evaporated to remove MeOH and TEA, and then dissolved in water. The sample was obtained by dialysis (MWCO=500 Da, 1 day) and lyophilization. 500 mg of lyophilized sample (0.511 mmol) was dissolved in 5 mL of MeOH and 0.53 mL of TEA (0.004 mol) was added into the sample solution. 18.4 mL of methyl acrylate (0.205 mol) was mixed with 13 mL of MeOH and then the sample solution was slowly added to the methyl acrylate solution over 30 min. The solution was reacted for 2 days at room temperature under a nitrogen atmosphere. After 2 days, to remove the MeOH and TEA, the solution was evaporated. The 2nd cystamine core was obtained by extraction with diethyl ether and water, and evaporation.

### 3rd Cystamine Core

3.24 g of cystamine dihydrochloride (14.40 mmol) and 4 mL of TEA (0.029 mol) were dissolved in 100 mL of MeOH. 300 mg of 2nd cystamine core (0.180 mmol) were dissolved in 5 mL of MeOH and then slowly added to the cystamine solution over 30 min. The solution was reacted for 4 days at 4° C. under a nitrogen atmosphere. After reaction, the mixture was evaporated to remove MeOH and TEA, then dissolved in water. The sample was obtained by dialysis (MWCO=1,000 Da, 1 day) and lyophilization. 300 mg of lyophilized sample (0.114 mmol) was dissolved in 5 mL of MeOH and 0.256 mL of TEA (0.002 mol) was added into the sample solution. 16.4 mL of methyl acrylate (0.183 mol) was mixed with 10 mL of MeOH and then the sample solution was slowly added to the methyl acrylate solution over 30 min. The solution was reacted for 2 days at room temperature under a nitrogen atmosphere. After 2 days, to remove the MeOH and TEA, the solution was evaporated. The 3rd cystamine core was obtained by extraction with diethyl ether and water, and evaporation.

7.3. PEI(s-s) 16 kDa and 32 kDa

The 2nd (0.060 mmol) and 3rd 10 (0.025 mmol) cystamine cores (100 mg of each) were dissolved in 5 mL MeOH. 8.6 g (4.8 mmole) and 7.2 g (4.0 mmole) of bPEI, 1.8 kDa and were each mixed with 50 mL MeOH, then 2nd and 3rd cystamine core solutions were slowly added to the 8.6 g and 7.2 g bPEI solutions over 30 min, respectively. The solutions were reacted for 5 days at room temperature under a nitrogen atmosphere. After 5 days, MeOH was removed by evaporation. The PEI(s-s) 16 kDa (2nd cystamine core with 8.6 g bPEI) and 32 kDa (3rd cystamine core with 7.2 g bPEI) were obtained by dialysis (MWCO=10,000 Da, 1 day), filtration (0.2 μm), and lyophilization. Each reaction step was monitored by Thin-Layer Chromatography and 1H nuclear magnetic resonance (118H NMR) (Bruker, 400 MHz). Branched polyethylenimine (bPEI 25 kDa, Mw 25,000 Da) was purchased from Sigma-Aldrich (St. Louis, Mo.). Branched polyethylenimine (bPEI 1.8 kDa, Mw 1,800 Da, bPEI 10 kDa, Mw 10,000 Da,) was purchased from Polysciences (Warrington, Pa.).

These newly designed and synthesized rPEIs are represented in FIG. 1. After synthesizing disulfide bond containing dendritic core, 1.8 kDa PEI was conjugated, and each reaction was analyzed by TLC and ¹H NMR. The occurrence of spectrum peaks of PEI(s-s)s were indicated as follows;

Cystamine dihydrochloride (400 MHz, D20): δ 3.05 (—SCH₂CH₂NH₃⁺), δ 3.42 (—SCH₂CH₂NH₃⁺).

Core molecule (400 MHz, MeOD): δ 2.48 (—NCH₂CH₂CO—), δ 2.80 (—SCH₂CH₂N—, —NCH₂CH₂CO—), δ 3.66 (—O—CH₃).

Bioreducible PEI (-s-s-) (400 MHz, D₂O): δ 2.28 (—NCH₂CH₂CO—; core), δ 2.45˜2.75 (—NCH₂CH₂N—; PEI), δ 2.98˜3.38 (—SCH₂CH₂N—, —NCH₂CH₂CO—; core).

In addition, the GPC results are indicated in Table 1. The measured molecular weights of PEI 1.8 kDa, rPEI 16 kDa, and rPEI 32 kDa were 1.8, 16, and 32 kDa, respectively. These molecular weights of polymers were consistent with theoretical values and it has narrow polydispersity index (P.D.). These NMR and GPC results showed that bioreducible PEI(s-s)s were successfully synthesized.

Disulfide bond crosslinking low molecular weight polymers can have improved and highly efficacious gene transduction efficacy with lower

Cytotoxicity as compared to 25 kDa PEI. Disulfide bonds in the polymer are quite stable in extracellular environment in vivo, yet they can be rapidly degraded in the presence of reductive glutathione inside the intracellular environment. In this regard, these newly synthesized bioreducible PEI can offer high gene transduction efficiency as well as low toxicity due to its reductive function.

TABLE 1
Molecular weights of rPEI polymers

| Polymer | Molecular weight |
|---|---|
| PEI 1.8 kDa | 1.8 kDa (P.D. = 1.14)^{a} |
| rPEI 16 kDa | 16 kDa (P.D. = 1.26)^{b} |
| rPEI 32 kDa | 32 kDa (P.D. = 1.24)^{b} |

^{a}Molecular weight as provided by the manufacturers.
^{b}Molecular weight was estimated by size-exclusion chromatography.

7.4. Cytotoxicity of Bioreducible Polymers

The 25 kDa PEI, 16 kDa rPEI or 32 kDa rPEI polymers were analyzed for cytotoxicity. The cell viability determination was performed by measuring the conversion of MTT to formazan as a function of time. A549, HT1080, and MCF7 cells were grown to 50% confluence in 96-well plates, then treated with varying polymer concentrations, up to 20 μg/mL. After 48 h, 50 μL of 3-(4,5-dimethylthiazol-2-yl)-2,5-diphenyl-tetrazolium bromide (MTT, 2 mg/mL in PBS; Sigma, St. Louis, Mo.) was added and incubated for 4 h at 37° C. The supernatant was removed, and the precipitate was dissolved in 200 μL dimethyl sulfoxide (DMSO; sigma). Plates were read on a microplate reader (Tecan Infinite M200; TecanDeutschland GmbH, Crailsheim, Germany) at 540 nm. The number of living cells in a PBS-treated cell group was analyzed for 100% cell viability.

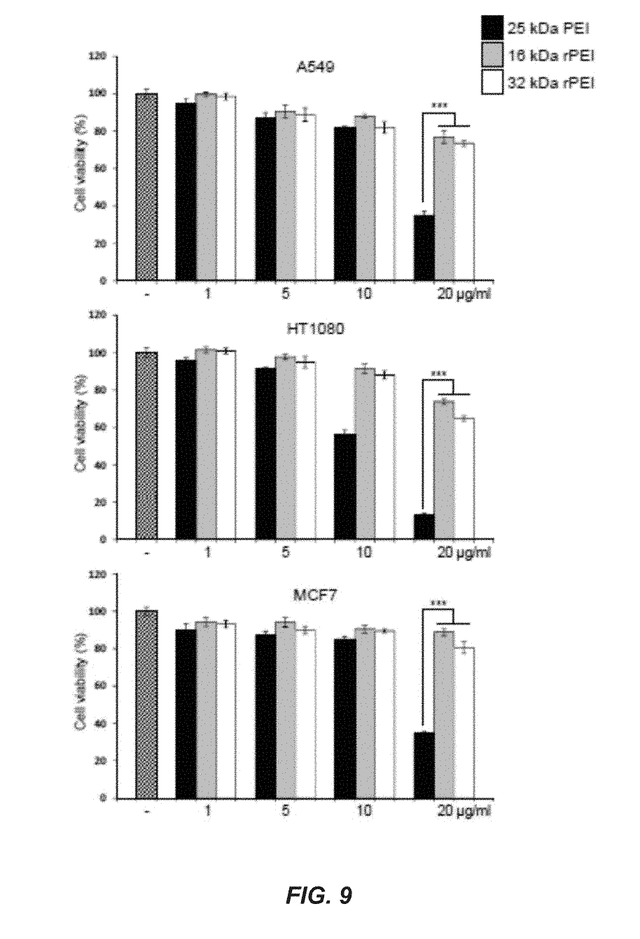

In order to evaluate the cytotoxicity of rPEIs, the cell viability of A549, HT1080, and MCF7 cells treated with rPEIs or 25 kDa PEI as a control were measured by MTT assays. As shown in FIG. 9, 25 kDa PEI exhibited significant toxicity and a much lower cytotoxicity was observed for rPEIs at concentrations below 20 μg/ml (***P<0.001). Meanwhile, 32 kDa rPEI exhibited more cytotoxicity than 16 kDa rPEI, but it still exhibited lower cytotoxicity than 25 kDa PEI at 20 μg/ml. These results may be attributed to the reducible property of rPEI which is easily degraded in an intracellular environment. Increased cytotoxicity of rPEI was observed with increasing molecular weight. Additionally, cationic polymers can induce cytotoxicity by interacting with intracellular components and higher molecular weight polymers have stronger interaction with these components, which results in higher toxicity. The viability of the cells treated with either of rPEI (16 kDa, 32 kDa) was above 76.8%, 73.4% for A549, 73.7%, 64.8% for HT1080, and 88.9%, 80.6% for MCF7 up to polymer concentrations of 20 μg/ml, respectively. These findings indicate that the reducible polymers can easily be dissociated inside the cytoplasm, and the results demonstrated much lower cytotoxicity than 25 kDa PEI.

7.5. Preparation of rPEI-Coated Adenovirus Complex and Size and Zeta Potentials

Complexes between cationic polymers and Ad particles (1×10¹⁰ VP) were formed by pre-diluting the PEI or rPEI cationic components and the Ad components in an E-tube using PBS (pH 7.4). The molar ratios of cationic molecules to Ad particle is described in the figure legends or methods section. The diluted rPEI polymers were added drop-wise to the solution of diluted Ad particles, mixed by inversion or tapping in a tube diluted to total volume of 100 μl with PBS solution. The Ad/polymer was allowed to complex at room temperature for 30 min through electrostatic interaction. The average sizes and zeta-potentials of naked Ad, Ad/25 kDa PEI, Ad/16 kDa rPEI, and Ad/32 kDa rPEI were measured with a Zetasizer 3000HS (Malvern Instruments, Inc., Worcestershire, UK) with a He—Ne laser beam (633 nm, fixed scattering angle of 90°) at 25° C. Ad particles (1×10¹⁰) were gently added to each polymer (1×10⁴ polymer molecules/Ad particle) diluted in PBS for 30 min. After the formation of complexes, PBS (pH 7.4) was added to a final volume of 1 mL. The obtained sizes and potential values are presented as the average values from three measurements.

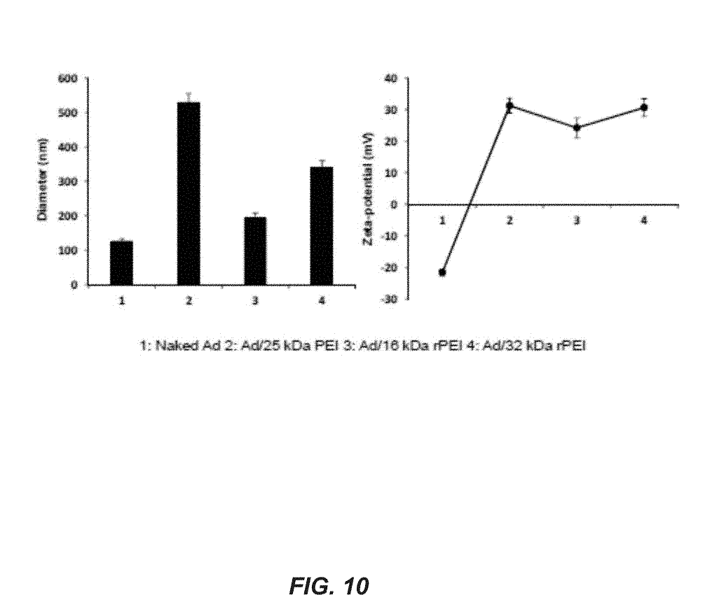

The formation of Ad/polymers nanoparticles was driven by electrostatic interaction between Ad and rPEIs. After generating Ad complexed with 25 kDa PEI or rPEIs the average size and surface charge of complexes were assessed by dynamic light scattering (DLS) and a zeta potential analyzer (FIG. 10). The diameter of the naked Ad was 125 nm; however, diameter of Ad/polymers was 529.1, 192.8, and 339.7 nm for 25 kDa PEI, 16 kDa rPEI, and 32 kDa rPEI, respectively. Additionally, the surface charge of the Ad/polymers complexes changed from a negative charge (−21.6 mV for naked Ad/GFP) to a positive with cationic polymers; 25 kDa PEI (31.2 mV), 16 kDa rPEI (24.3 mV), and 32 kDa rPEI (30.6 mV). Interestingly, Ad complexed with either rPEI exhibited lesser diameter than Ad/25 kDa PEI due to increased condensation between Ad and rPEIs meanwhile surface charge of Ad/rPEIs was comparable to Ad/25 kDa PEI. These results indicated that positively charged 16 kDa rPEI polymer to viral particles produced a particle diameter of approximately 200 nm with cationic surface which suggests that Ad/rPEI can be efficiently transduced into cells.

7.6. Cellular Uptake of Ad/Polymers

The naked Ad was conjugated with Fluorescein isothiocyanate (FITC, Sigma) in 1 mL PBS for 4 h, then Ad-FITC was dialyzed (10K cut off, Slide-A-Lyzer™ Dialysis Cassettes, Life Technologies, Grand Island, N.Y.) to remove unconjugated FITC at 4° C. in a cold room. A549, or HT1080 cells were plated onto 24-well plates at about 70-80% confluence. After 24 h, cells were treated with Ad-FITC, Ad-FITC/25 kDa PEI, Ad-FITC/16 kDa rPEI, and Ad-FITC/32 kDa rPEI multiplicity of infection (MOI) of 200 (A549), 500 (HT1080) for 2 h, then washed with ice cold-PBS three times. Cellular uptake activity was quantified by measuring the fluorescence intensity with Tecan Infinite M200.

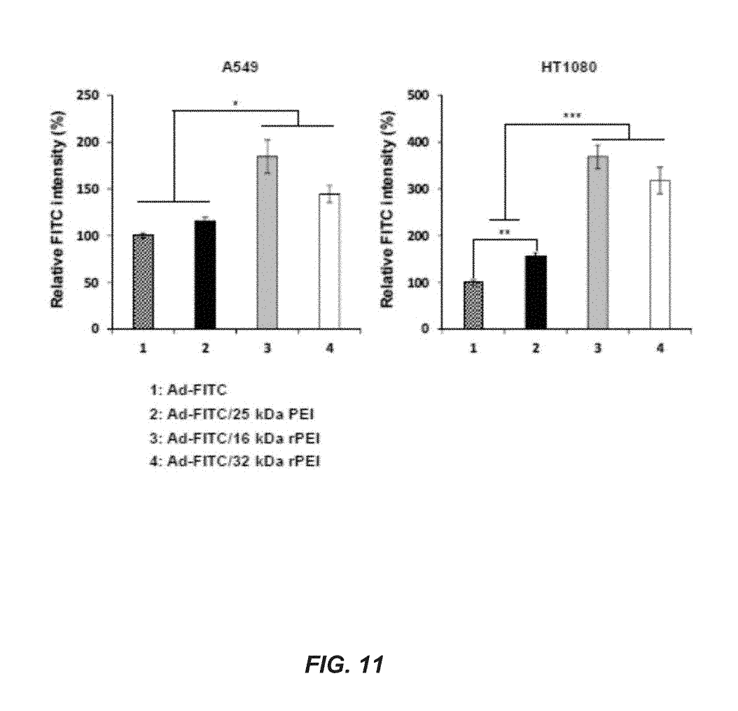

FIG. 11 shows the cellular uptake efficiency of Ad conjugated with FITC complexed with 25 kDa PEI or rPEIs. The cellular uptake was markedly enhanced when Ad was complexed with either rPEIs compared to naked Ad or Ad/25 kDa PEI (*P<0.05 for A549, ***P<0.001 for HT1080). 16 kDa or 32 kDa rPEI-coated Ad enhanced the uptake by 1.6- or 1.2-folds in A549 with 2.4- or 2.0-fold higher uptake in HT1080 than Ad/25 kDa PEI, respectively. These results indicate that size of Ad/nanocomplex also contributes to the efficiency of cellular uptake. There are two key determining factors of gene transduction efficiency; 1) cellular uptake and 2) efficient endosomal escape. Internalization of Ad complexed with cationic polymer differs greatly from naked Ad as cellular uptake mechanism of Ad is CAR-mediated endocytosis while Ad/polymer's uptake is dependent on cationic surface charge of the polymer and polymer with stronger positive charge can be internalized more efficiently by macropinocytosis through interacting with negatively charged cellular membrane. This evaluation demonstrates that cellular uptake of Ad/rPEIs is more efficient than conventional Ad uptake pathway. Of note, size of Ad/cationic polymers is also integral for cellular uptake as polymers with similar cationic charge are heavily influenced by size, as rPEIs which have similar charge but smaller size than 25 kDa PEI showed significantly enhanced cellular uptake. Furthermore, PEIs are well known for high endosomal escape ability through proton sponge effect, which contributes to high gene transduction efficiency which must also contribute to transduction efficacy of rPEIs. These results in conjunction strongly suggest that Ad complexed with either rPEIs have high transduction activity due to rPEIs' surface charge, size and high endosome escape capacity.

7.7. Transduction Efficiency Assay

Transduction efficiency of naked Ad/GFP, Ad/GFP/25 kDa PEI, Ad/16 kDa rPEI or Ad/32 kDa rPEI was assessed by measuring GFP expression in both CAR-positive (A549) and CAR-low, negative (HT1080 and MCF7) cells. Cells were seeded at a density of 5×10⁴ cells/well in 24-well plates. After 24 h, cells were treated with Ad/GFP, Ad/GFP/25 kDa PEI, Ad/16 kDa rPEI or Ad/GFP/32 kDa rPEI at 20 (A549), 100 (HT1080) or 300 (MCF7) MOI. After 48 h of incubation, cells were observed by fluorescence microscopy (Olympus IX81; Olympus Optical, Tokyo, Japan). For quantifying adenoviral transduction, GFP expression levels were quantified by measuring the fluorescence at 485 nm for excitation and 535 nm for emission in a plate reader (Tecan Infinite M200), and were indicated as values of mean fluorescence intensity (MFI).

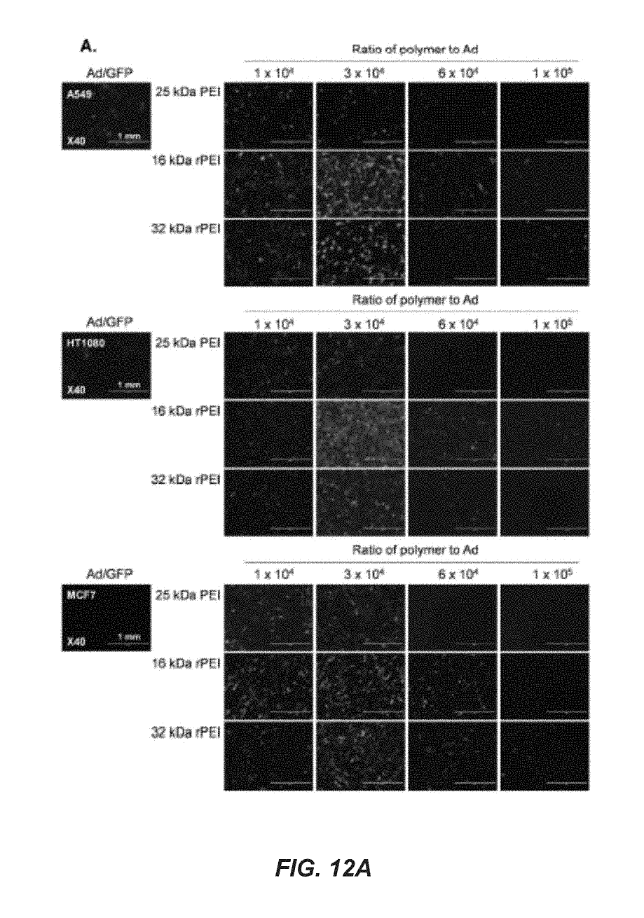

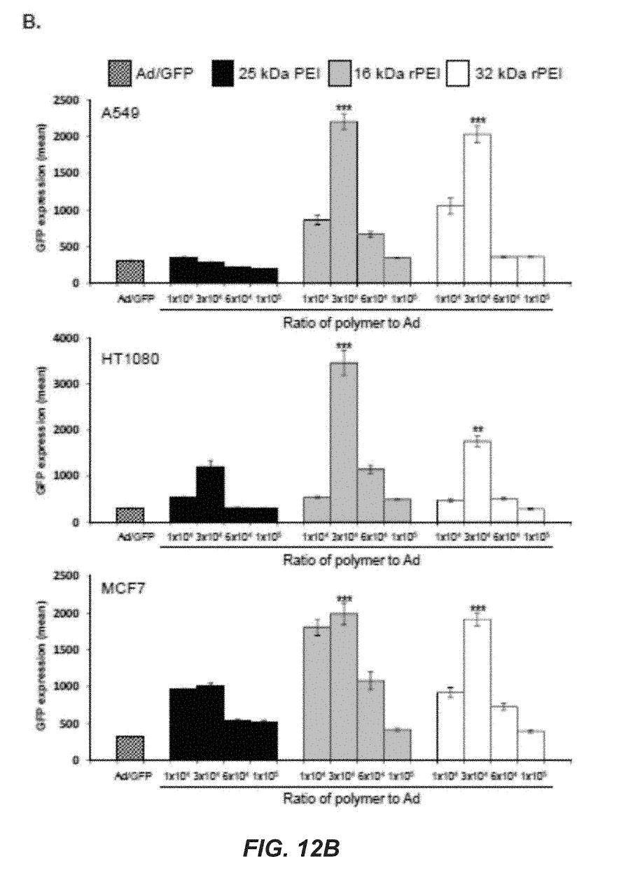

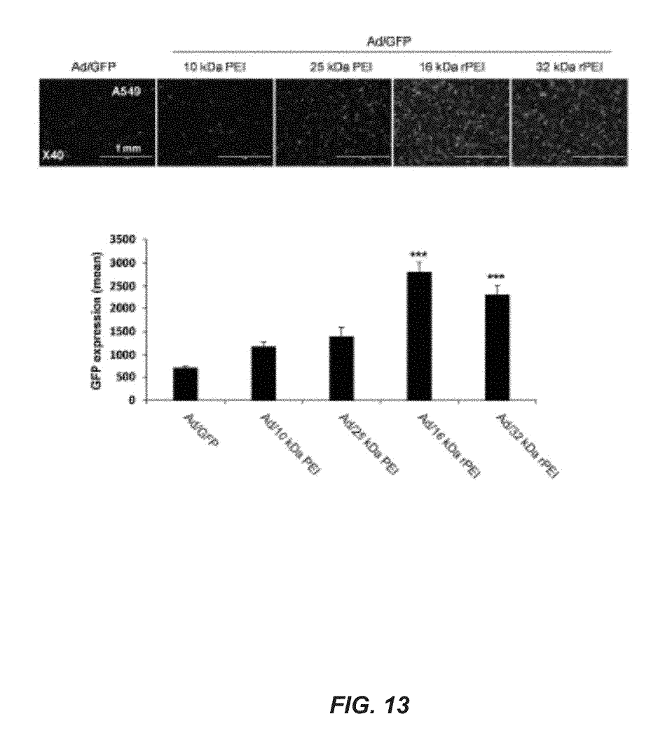

Naked Ad transduction is highly dependent on CAR expression level of cellular membrane. Malignant tumors often have diminished or ablated CAR expression, which nullifies Ad tumor infectivity which contributes to low therapeutic efficacy. One strategy to overcome CAR dependency is to utilize hybrid vector of Ad and cationic polymer as it can be internalized by CAR-independent macropinocytosis. To evaluate Ad/rPEI's CAR4 independent transduction, CAR-positive A549, low CAR expressing HT1080, and CAR5 negative MCF7 cells were transduced with Ad/rPEI or Ad/25 kDa PEI. The transduction efficiency of Ad/rPEIs was markedly increased compared to naked Ad in A549, HT1080, and MCF7 cells (FIG. 12). This suggests that Ad/rPEIs can efficiently transduce cancer cells independent of CAR expression. Importantly, the beneficial effect of rPEIs complexation were particularly pronounced in CAR-low (HT1080) and -negative (MCF7) cells where the transduction efficiency was increased by 11.7- and 5.9-fold (3×10⁴ 16 kDa rPEI, ratio of rPEI to Ad) and 5.7 and 6.0-fold (1×10⁴ 32 kDa, ratio of rPEI to Ad) compared to naked Ad, respectively (***P<0.001). More importantly at 3×10⁴ 16 kDa rPEI or 32 kDa rPEI ratio of rPEI to Ad, GFP expression was 7.7- and 7.1-fold higher in A549 cells, 2.9- and 1.5-fold higher in HT1080 cells, and 2.0- and 1.9-fold higher in MCF7 cells treated with Ad/rPEIs than Ad/25 kDa PEI which demonstrated superior transduction efficiency of rPEIs in both CAR-positive and -negative cells. Moreover, we evaluated transduction efficiency of adenovirus complexed with 10 kDa PEI; 10 kDa PEI had closest molecular weight to our primary polymer of interest (rPEI 16 kDa). As shown in FIG. 13, Ad/GFP/10 kDa PEI treated cells showed higher GFP expression levels than naked Ad/GFP treated cells, yet its GFP expression was lower than those complexed with 25, 32 kDa PEI, or 16 kDa rPEI. This result suggests that 16 kDa rPEI is more efficient carrier of adenovirus than 10 kDa PEI (commercial PEI of similar kDa as rPEI used) and 25 kDa PEI which is known as the “gold standard” of polymer-based gene delivery system. Gene transduction efficiency can be decided by optimal molar ratio of polymer to Ad. Additionally, cellular uptake of Ad/polymer complex can be mediated by clathrin, caveolin-mediated endocytosis or micropinocytosis. This indicates that cellular uptake mechanism of Ad/polymer differs from that of naked Ad. Size and surface charge of nanoparticles are crucial factor for passively targeted delivery, which is affected by polymer/Ad molar ratio. Furthermore, excess polymers can induce aggregation when complexed with adenovirus, which prevents efficient cellular uptake as well as endosomal escape, resulting in decreased transduction efficiency. Ad/polymer complex's transduction efficiency is heavily dependent on ratio of polymer to Ad. Interestingly, quantity of polymer used for optimal ratio of Ad/rPEIs (16 kDa—1×10⁴; 32 kDa—3×10⁴) can be approximately 100-fold less than other polymers complexed with Ad, such as ABP or PNLG. This suggests that low amount of rPEI can efficiently encapsulate the Ad resulting in high gene transduction with low toxicity.

7.8. Competition Assay

A549 cells were seeded in 24-well plates at 5×10⁴ cells per well. 24 h later, cells were pretreated with CAR antibody (20, 50 μg/ml) in serum-free DMEM for 30 min. Naked Ad/GFP, Ad/GFP/25 kDa PEI, Ad/GFP/16 kDa rPEI, and Ad/GFP/32 kDa rPEI were added with MOI of 50 to each well and incubated at 37° C. 48 h later, cells were observed by fluorescence microscopy (Olympus BX51). GFP expression levels were also quantified by fluorescence intensity (Tecan Infinite M200).

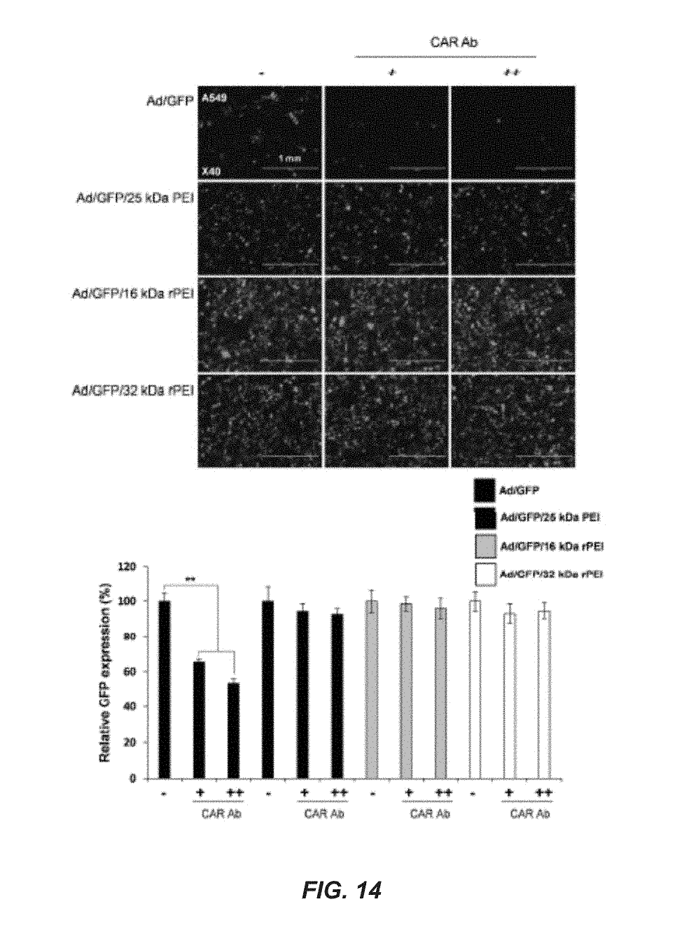

The competition assay was performed using CAR specific antibody (Ab) to demonstrate that Ad/polymer is internalized independently of CAR-mediated endocytosis. CAR-positive A549 cells were pre-incubated with CAR-specific Ab prior to transduction with replication-incompetent GFP expressing Ad. GFP expression of naked Ad/GFP was substantially reduced in a dose-dependent manner for amount of CAR-specific Ab treated to cells prior to transfection (35% and 47% decreases with 20 and 50 μg/ml CAR Ab pretreatment, respectively; FIG. 14). In contrast, GFP expression of Ad/GFP complexed with cationic polymers was not blocked by CAR Ab, suggesting that Ad/GFP/polymers' uptake pathway was not mediated by interaction between adenoviral fiber and CAR. This data shows that uptake of Ad/rPEIs is internalized by alternative pathway from traditional CAR-mediated endocytosis pathway which make Ad/rPEIs feasible for treatment of malignant tumors regardless of their CAR expression.

7.9. Cancer Cell Killing Activity

A549, HT1080, and MCF7 cells grown to 50% confluence in 24-well plates were transduced with naked oncolytic RdB/shMet (oAd), oAd/25 kDa PEI, oAd/16 kDa rPEI, and oAd/32 kDa rPEI at an MOI of 5 (A549), 50 (HT1080), and 200 (MCF7), respectively. Two days after infection, 250 μl MTT (Sigma, 2 mg/mL) was added to each well. The cells were incubated at 37° C. for 4 h, and the supernatant was then discarded. The precipitate was dissolved in 1 mL DMSO (Sigma), and the plates were then read with a microplate reader at 540 nm. The number of living cells in a PBS-treated group was analyzed as 100% viability. For viral production assay, A549, HT1080, and MCF7 cells were transduced in the same condition as above for 4 h. Then cells were washed with PBS and changed with 5% FBS contained DMEM. After four days, infectious Ad viral particles were determined by limiting dilution assay.

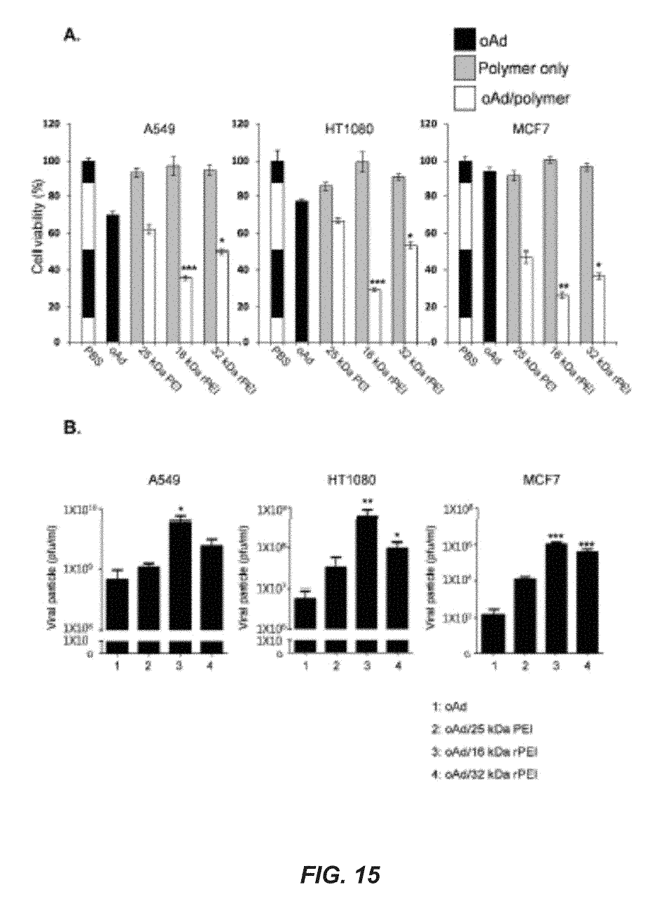

The oncolytic Ad (RdB/shMet or oAd) was genetically designed and produced to selectively replicate in and kill cancer cells by tumor specific promoter. This E1A- and E1B-double mutated promoter inserted oncolytic Ad can tumor selectively produce the viral progeny and ultimately infect neighboring cancer cells. The signaling of the c-Met and its ligand, HGF, is deregulated in many cancers, and is known to critically play a role affecting both primary tumor growth and metastasis. And thus, Ad expressing shMet, which downregulates c-Met, can be a new targeted therapeutic approach for cancer gene therapy. To investigate whether the oncolytic effect of oAd coated with rPEIs could be improved, oAd was physically coated with rPEIs and treated to the cancer cells (FIG. 15A). MTT assay revealed that naked oAd killed 30.3% (A549), 22.8% (HT1080) or 5.6% (MCF7) which is significantly less than killing efficacy observed by Ad/polymers as even Ad/25 kDa PEI which showed lowest cytotoxicity of Ad/polymers exhibited 38% (A549), 33.4% (HT1080), or 53.5% (MCF7); Ad/16 kDa or 32 kDa rPEI exhibited greater cytotoxicity in all cell lines 64.3%, 50.3% (A549), 70.9%, 46.7% (HT1080) and 73.8%, 63.4% (MCF7). These results showed that cancer cell killing effect of oncolytic Ad can be significantly improved by coating with rPEIs in both CAR-positive and -negative cancer cells. As shown in FIG. 9, rPEIs polymers toxicity did not significantly affect cell killing effect, so a viral production assay was performed to evaluate the virus' enhanced cancer cell killing efficacy (FIG. 15B). A549, HT1080, and MCF7 cells were treated with oAd, oAd with 25 kDa PEI, 16 kDa rPEI, or 32 kDa rPEI for 4 h and then media was changed to remove any polymers and measure quantity of viral progeny. As shown in FIG. 15B, the viral production assay result of oAd or oAd/polymer complex was strongly correlated with that of cancer cell killing effect (FIG. 15A). The mechanism of cancer cell killing effect by oncolytic Ad is highly dependable on its lytic cycle through production of viral progeny as well as expressing therapeutic gene. Since CAR expression levels can differ in each cell line, treatment of different MOI is needed for certain cell lines to show efficient transduction. The purpose of viral production assay is observation of viral progeny, which is decided by initial infection, and for this procedure treating with significant surplus of oncolytic Ad can induce apoptosis before viral progeny assembly that decreases viral production. As optimal condition and dose of oncolytic Ad varies significantly between each cell line, different MOI was utilized in correlation with relative CAR expression. More specifically, oAd/16 kDa rPEI produced 8.9-, 5.5-, and 2.4-fold (A549), and 107-, 18-, and 6.3-fold higher viral production than oAd-, oAd/25 kDa PEI-, or oAd/32 kDa rPEI-treated cells, respectively. rPEIs did not affect viral production and oAd/16 kDa rPEI polymer showed strongest cancer-killing effect with highest viral production in the cells. These data showed despite polymer only helps with initial viral entry, but it is important to note that more viral progenies are produced due to higher cellular uptake during initial entry as more cells are infected. After Ad infect to the cells, 1000˜10,000 viral progeny is produced and sequentially infect to the surrounding cancer cells. Improved initial transduction can significantly enhance cancer treatment as lower initial dose of virus can be administered, and charge-mediated entry can show substantial difference in therapeutic outcomes in treatment of CAR-ablated or low-expressing cancers. For example cancer cells were infected with 50 MOI of oncolytic Ad/polymer can get similar therapeutic effects as naked oncolytic Ad administered at MOI of 50,000˜500,000. Moreover, reduced viral dose per administration can be advantageous in terms of safety. Thus combination of oncolytic Ad vector and non-viral vector give rise to increase therapeutic effect as well as safety.

7.10. Measurement of Met or VEGF Expression

A549 and HT1080 cells were seeded on 6-well plate with a number of 1×10⁵ cells per well. After 24 h, the plated cells were treated with oAd, oAd/25 kDa PEI, oAd/16 kDa rPEI, and oAd/32 kDa rPEI at an MOI of 2 (A549), and 20 (HT1080), respectively. Three days later, each conditioned medium was harvested and the expression level of Met or VEGF was measured by Met (Invitrogen, Life technologies, Grand Island, N.Y.) or VEGF (R&D Systems, Inc., Minneapolis, Minn.) ELISA assay kit. Measured-value was normalized with cell lysate.

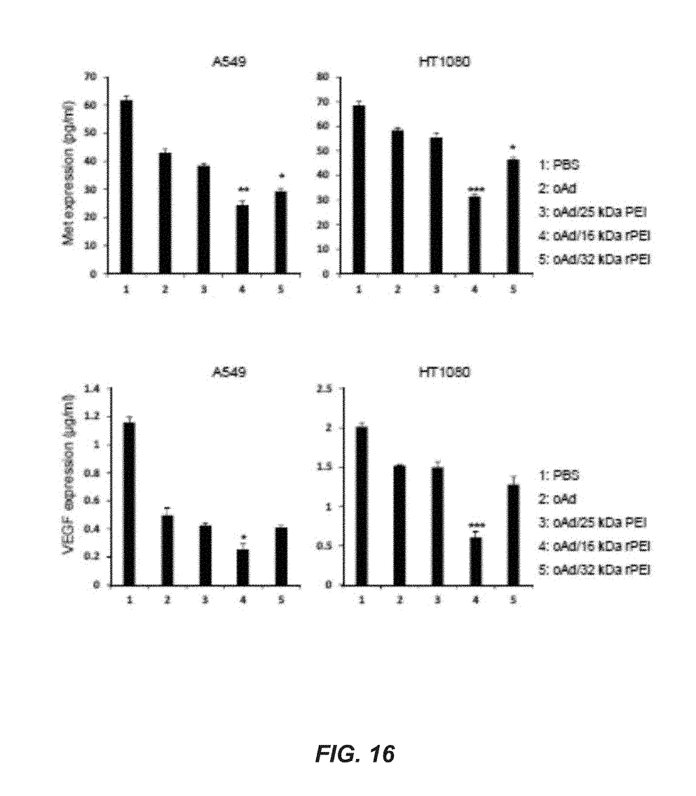

Downregulation of Met signaling has high therapeutic potential against cancer as it can suppress cancer cell migration and invasion as well as inhibiting both tumor growth and angiogenesis. In theory shMet, which can downregulate Met signaling, should be highly beneficial, but shMet as a single therapeutic agent has limitation due to short half-life of siRNA. In this study, shMet was genetically inserted into oncolytic Ad backbone to increase quantity of shMet delivered and expressed in tumor as each replication and lytic cycle of Ad would amplify expression of shMet as Ad can produce approximately 1000˜10000 copies of viral progenies which can sequentially infect neighboring cancer cells. This resulted in both high and prolonged expression of shMet overcoming the limitation of therapy based on siRNA as single therapeutic agent. To further evaluate the effect of shMet to suppress the expression Met and VEGF, the secretion level of Met and VEGF were determined. As shown in FIG. 16, the expression of Met and VEGF was significantly suppressed in the cells treated with oAd/16 kDa rPEI complex in comparison to oAd- or oAd/25 kDa PEI-treated cells. Furthermore, the secretion of Met from the oAd/16 kDa rPEI-treated A549 or HT1080 cells was inhibited by 60.7% or 54.2%, respectively, whereas the expression of Met or VEGF expression was decreased by lesser extent by other treatment groups; 30.68% or 14.5% for naked oAd, 37.9% or 19% for oAd/25 kDa PEI, and 52.6% or 32.4% for oAd/32 kDa rPEI, respectively. Meanwhile, the secretion of VEGF from the oAd/16 kDa rPEI-treated A549 or HT1080 cells was inhibited by 79.2% or 70%, respectively, whereas the expression VEGF expression was decreased by 58.3% or 25% for naked oAd, 65% or 26% for oAd/25 kDa PEI, and by 65.8% or 36.5% for oAd/32 kDa rPEI, respectively. Taken together, these data suggest that the oAd did not affect the functionality of Ad after complexation with any of the polymers used in this study. Conclusively, oAd/rPEI nanocomplex could maximize the cancer cell killing ability through both adenoviral oncolysis and suppression of Met and VEGF expression in broad range of cancer without significant polymer toxicity. However, poor tumor selectivity could be a potential disadvantage for complexation of Ad with a cationic polymer as strong cationic surface charge of the complex can result in indiscriminate cellular uptake of complex even by normal cells due to charge mediated internalization of the complex into negatively charged membranes. In order to overcome such limitations, the outer shell or distal end of polymer can be conjugated with active targeting moieties such as antibodies, growth factors, small peptides, or ligands.

7.11. Statistical Analysis

The data in Example 7 are expressed as mean+/− standard deviation (SD). Statistical comparisons were performed with Stat View software (Abacus Concepts, Inc., Berkeley, and Mann-Whitney tests (nonparametric method). The criterion for statistical significance was *P<0.05.

While the forgoing examples are illustrative of the specific embodiments in one or more particular applications, it will be apparent to those of ordinary skill in the art that numerous modifications in form, usage and details of implementation can be made without departing from the principles and concepts articulated herein. Accordingly, no limitation is intended except as by the claims set forth below.

1. A polymeric carrier for delivery of a payload to a cell, comprising:
a biodegradable core molecule having a disulfide bond, an amide group, and a tertiary amine; and
a poly(ethylenimine) (PEI) connected to the biodegradable core molecule.

2. The polymeric carrier of claim 1, wherein greater than 50% of biodegradable bonds in the biodegradable core molecule are disulfide bonds.

3. The polymeric carrier of claim 1, wherein the biodegradable core molecule comprises from 1 to 13 disulfide bonds.

4. The polymeric carrier of claim 1, wherein the biodegradable core molecule has from 4 to 28 amide groups.

5. The polymeric carrier of claim 1, wherein the biodegradable core molecule has from 2 to 14 tertiary amines.

6. The polymeric carrier of claim 1, wherein the biodegradable core molecule is a dendrimer.

7. The polymeric carrier of claim 6, wherein the dendrimer has from 4 to 16 termini.

8. The polymeric carrier of claim 1, wherein the PEI is a linear PEI.

9. The polymeric carrier of claim 1, wherein the PEI is a branched PEI.

10. The polymeric carrier of claim 1, wherein the PEI has a molecular weight of between 1.0 and 3.0 kilodaltons (kDa).

11. The polymeric carrier of claim 1, having a molecular weight of between about 7.4 and 7.8 kDa.

12. The polymeric carrier of claim 1, having a molecular weight of between about 15.5 and 16.5 kDa.

13. The polymeric carrier of claim 1, having a molecular weight of between about 31.5 and 32.5 kDa.

14. The polymeric carrier of claim 1, having a polydispersity of from 1.20 to 1.26.

15. The polymeric carrier of claim 1, having a transfection efficiency greater than 60%.

16. (canceled)

17. (canceled)

18. (canceled)

19. (canceled)

20. A non-viral delivery system for delivery of a payload to a cell, comprising:
a polymeric carrier, the polymer carrier comprising:
a biodegradable core molecule having a disulfide bond, an amide bond, and a tertiary amine, and
a poly(ethylenimine) (PEI) connected to the biodegradable core molecule; and
a non-viral payload.

21. The non-viral delivery system of claim 20, wherein the non-viral payload is a member selected from the group consisting of: DNA, RNA, a protein, a therapeutic agent, and combinations thereof.

22. (canceled)

23. The non-viral delivery system of claim 20, wherein a weight ratio of the non-viral payload to polymeric carrier is from about 1:1 to about 1:10.

24. The non-viral delivery system of claim 20, wherein the polymeric carrier has a molecular weight of between about 15.5 and 16.5 kDa.

25. (canceled)

26. (canceled)

27. (canceled)

28. (canceled)

29. (canceled)

30. A method of treating a disease in a subject, comprising:
administering a therapeutic gene to a target cell of a subject via a polymeric carrier according to claim 1.

31. (canceled)

32. (canceled)

33. (canceled)
